(19) United States
(12) Patent Application Publication
(10) Pub. No.: US 2013/0158070 A1
(43) Pub. Date: Jun. 20, 2013

(54) CONJUGATES OF POLYUNSATURATED FATTY ACIDS AND AMINE-CONTAINING COMPOUNDS AND USES THEREOF

(71) Applicant: Taher Nassar, Tur'an (IL)

(72) Inventor: Taher Nassar, Tur'an (IL)

(21) Appl. No.: 13/820,678

(22) PCT Filed: Sep. 6, 2011
(86) PCT No.: PCT/IL11/00708

§ 371 Date: Mar. 4, 2013

Related U.S. Application Data
(60) Provisional application No. 61/380,272, filed on Sep. 6, 2010.

(51) Int. Cl. C07D 207/16; C07C 233/55; C07D 498/04; C07D 213/80; C07D 209/26; C07C 309/13; C07C 69/533

(52) U.S. Cl. CPC C07D 207/16; C07C 309/13; C07C 233/55; C07C 69/533; C07D 213/80; C07D 209/26; C07D 498/04
USPC 514/302; 514/423; 548/533; 554/44; 514/578; 514/563; 554/63; 554/223; 514/548; 546/309; 514/352; 548/500; 514/419; 546/115

(57) ABSTRACT

Novel chemical conjugates derived from unsaturated fatty acids and therapeutically active agents, are disclosed. The chemical conjugates are designed and characterized as COX-2 and/or 5-LOX inhibitors and are useful in the treatment of inflammatory diseases and disorders such as Alzheimer's disease, Parkinson's disease, asthma, osteoarthritis, rheumatoid arthritis, pain, primary dysmenorrhea, Crohn's disease and ulcerative colitis.

### FIELD AND BACKGROUND OF THE INVENTION

The present invention, in some embodiments thereof, relates to chemical conjugates and more particularly, to conjugates of a fatty acid and a therapeutically active agent, which can be used as COX-2 inhibitors and optionally also as 5-LOX inhibitors, for treating inflammation.

Inflammation is a self-defensive reaction aimed at eliminating or neutralizing injurious stimuli, and restoring tissue integrity. Inflammation is mediated by hormone-like compounds called prostaglandins, which cause inflammation and pain. Cyclooxygenases (COXs) are enzymes responsible for forming prostaglandins in the body. Non-steroidal anti-inflammatory drugs (NSAIDs), which inhibit COX, are effective at reducing inflammation, but cause unwanted side effects.

There are two cyclooxygenase (COX) enzymes at work in the body, COX-1 and COX-2. The COX-1 enzyme is expressed in most tissues, and produced widely throughout the body, and is necessary for a variety of important internal housekeeping functions, such as protecting the stomach lining, maintaining renovascular function and platelet aggregation, and is involved in the regulation of day-to-day cellular and metabolic activities such as maintaining stomach lining integrity, regulating blood flow within the kidneys and balancing platelet function. In contrast, COX-2 enzyme is an “inducible” isoform, expressed in response to a variety of pro-inflammatory stimuli and found in the brain, male and female reproductive organs, kidneys and in bone-forming cells called osteoblasts. Unlike COX-1, COX-2 expression is usually minimal, but when activated, COX-2 regulates prostaglandin production primarily within inflammatory cells. This inflammatory response is a vital part of healing and repairing.

Although NSAIDs are effective, they inhibit both COX-2 and COX-1. This is problematic because COX-1 inhibition interferes with important functions such as the repair and maintenance of stomach lining, and may therefore result in varying degrees of gastric ulcerations, perforations or obstructions in one-third to almost one-half of patients administered with COX-1 inhibiting NSAIDs. Hence, there has been considerable interest in the development of selective COX-2 inhibitors, such as the COX-2 inhibitors celecoxib and rofecoxib.

Omega-3 fatty acids, such as those found in fish oils, have been recommended for managing chronic inflammatory conditions given their ability to alter prostaglandin production and to yield measurable changes in certain disease parameters in rheumatoid arthritis patients. Clare Curtis and colleagues have reported that omega-3 fatty acids (and not other fatty acids) dose-dependently inhibited production of COX-2 expression without affecting COX-1 expression in an in vitro model, and inhibited degradation of aggrecan, a hallmark process of arthritic conditions.

Flavonoids are a class of plant-derived chemicals that have been investigated for anti-inflammatory effects. Five flavonoids, genistein, kaempferol, quercetin, resorcinol and resveratrol have been reported to produce dose-dependent decreases in TGF-α-induced COX-2 activity, with quercetin being the most potent [Mutoh et al. Jpn J Cancer Res. 2000 July; 91(7):686-91].

Resveratrol has also been reported by researchers from Cornell Medical College to inhibit COX-1 and COX-2 activity in mammary and oral epithelial cells [Subbaramaiah et al., J Biol Chem 1998, 273:21875-21882; Zewczuk et al. J Biol Chem. 2004 May 21; 279 (21):22727-37]. Another study has reported that resveratrol inhibits COX-2 expression in mouse macrophages without affecting COX-1 protein expression [Martinez & Moreno, Biochem Pharmacol 2000, 59:865-870]. However, an additional study found no effect of resveratrol on COX-2 induction in mouse skin Pang & Pezzuto, Cancer Lett 1998, 134:81-89].

Inflammation occurs in pathologically vulnerable regions of the Alzheimer's disease (AD) brain. In the AD brain, damaged neurons and neurites and highly insoluble amyloid β peptide deposits and neurofibrillary tangles provide potential stimuli for inflammation. Thus, animal models and clinical studies, although still in their infancy, suggest that inflammation in the AD brain significantly contributes to AD pathogenesis.

In Parkinson's disease, postmortem examination reveals a loss of dopaminergic neurons in the substantia nigra associated with a massive astrogliosis and the presence of activated microglial cells. Recent evidence suggests that the disease may progress even when the initial cause of neuronal degeneration has disappeared, suggesting that toxic substances released by the glial cells may be involved in the propagation and perpetuation of neuronal degeneration [Hirsch et al., Ann N Y Acad Sci. 2003 June; 991:214-28]. Glial cells can release deleterious compounds such as proinflammatory cytokines (TNF-α, Il-1β, IFN-γ), which may act by stimulating nitric oxide production in glial cells, or which may exert a more direct deleterious effect on dopaminergic neurons by activating receptors that contain intracytoplasmic death domains involved in apoptosis. The anti-inflammatory drugs pioglitazone, a PPAR-γ agonist, and the tetracycline derivative minocycline, have been shown to reduce glial activation and protect the substantia nigra in an animal model of the disease degeneration has disappeared, suggesting that toxic substances released by the glial cells may be involved in the propagation and perpetuation of neuronal degeneration [Breidert et al. Proc. Natl. Acad. Sci. USA 2002, 98: 14669-14674].

Inflammation is an essential part of the functioning of a normal lung. Tiny areas of inflammation, typically via IgE antibodies, occur thousands of times a day in order to combat the viruses, bacteria and pollutants to which lungs are exposed. Normally, none of this activity produces any obvious symptoms. However, asthmatics react excessively to some factors, leading to aggravated inflammation throughout the small and medium airways. It is thought that asthmatics over-produce unique IgE antibodies in response to these factors.

International Patent Application PCT/IL2007/001592 (published as WO 08/075366) describes conjugates of fatty acids with amines and uses thereof in inhibiting cyclooxygenase enzymes and treating inflammations.

U.S. Pat. No. 4,933,324 discloses a prodrug comprising a fatty acid carrier such as 4,7,10,13,16,19-docosahexa-enoic acid covalently bound to a neuroactive drug such as dopamine.

U.S. Pat. No. 5,300,665 discloses a process for preparing fatty acid esters of hydroxyalkylsulfonates and fatty acid amides of aminoalkylsulfonates.

Additional background art includes U.S. Pat. No. 4,218,404, U.S. Pat. No. 4,443,475, U.S. Pat. No. 7,034,058, and International Patent Application PCT/IN2009/000382 (published as WO 2010/004579).

Recently, a therapeutic role of dual inhibitors of COX and 5-LOX has been suggested. For a detailed discussion in this regard see, Martel-Pelletier et al., Ann Rheum Dis 2003 62: 501-509. While both the conventional NSAIDs and the selective COX-2 inhibitors primarily exert their activity by reducing the production of PGs induced in the inflammatory process, in recent years, it has been clarified that PG synthesis is only one part of the arachidonic acid pathway, this precursor being a substrate that gives rise to many other lipid mediators, such as the LTs and the LXs.

Leucotrienes themselves have a major role in the development and persistence of the inflammatory process, and it is now clear that PGs and LTs have complementary effects, whereas the production of LXs can counteract the inflammatory actions of LTs. In view of these concepts, it has been suggested that blocking both LT and PG production might have synergistic effects and achieve optimal anti-inflammatory activity. In addition, taking into account the roles of LTB4 and cysteinyl LTs (against which neither selective nor non-selective NSAIDs are effective, in the inflammatory process, dual inhibition of the COX and 5-LOX pathways could produce a wider spectrum of anti-inflammatory effects. Dual inhibition of COX and 5-LOX may limit the vascular changes seen during inflammation and leucocyte induced GI damage.

### SUMMARY OF THE INVENTION

The invention provides some structural and functional features of chemical conjugates of a therapeutically active agent and a hydrophobic moiety, which impart to the conjugates an efficient and selective COX-2 inhibitory activity, and optionally also a 5-LOX inhibition activity. Some of the currently disclosed conjugates employ known anti-inflammatory drugs conjugates to hydrophobic moieties such as unsaturated fatty acids, while some employ medical food agents.

According to an aspect of some embodiments of the present invention there is provided a chemical conjugate comprising a first moiety and a second moiety covalently linked therebetween, wherein the second moiety is derived from docosa-4,7,10,13,16,19-hexaenoic acid, and wherein the first moiety is derived from a therapeutically active agent or a derivative thereof, each independently having a functional group for forming a covalent bond with the second moiety, with the proviso that the first moiety is not hydroxyproline, the chemical conjugate being a cyclooxygenase-2 (COX-2) inhibitor.

According to some embodiments of the invention, the chemical conjugate is further capable of inhibiting 5-lipoxygenase (5-LOX) inhibitor.

According to some embodiments of the invention, the functional group is selected from the group consisting of hydroxy, amine, carboxy and amide.

According to some embodiments of the invention, the first moiety and the second moiety are covalently bound via a bond selected from the group consisting of an amide bond and an ester bond.

According to some embodiments of the invention, the therapeutically active agent is an anti-inflammatory agent.

According to some embodiments of the invention, the therapeutically active agent is a cyclooxygenase (COX) inhibitor.

According to some embodiments of the invention, the therapeutically active agent is a non-steroidal anti-inflammatory drug (NTHE).

According to some embodiments of the invention, the therapeutically active agent is selected from the group consisting of: 5-hydroxy-indol-3-yl-acetic acid, 2-amino-nicotinic acid, salicyclic acid, mesalazine and quercetin.

According to an aspect of some embodiments of the present invention, there is provided a chemical conjugate comprising a first moiety and a second moiety covalently linked therebetween, wherein the second moiety is derived from y-linolenic acid, and wherein the first moiety is derived from a therapeutically active agent or a derivative thereof, each independently having a functional group for forming a covalent bond with the second moiety, with the proviso that the first moiety is not hydroxyproline or taurine, the chemical conjugate being a cyclooxygenase-2 (COX-2) inhibitor.

According to some embodiments of the invention, the chemical conjugate is further capable of inhibiting 5-lipoxygenase (5-LOX).

According to some embodiments of the invention, the functional group is selected from the group consisting of hydroxy, amine, carboxy and amide.

According to some embodiments of the invention, the first moiety and the second moiety are covalently bound via a bond selected from the group consisting of an amide bond and an ester bond.

According to some embodiments of the invention, the therapeutically active agent is an anti-inflammatory agent.

According to some embodiments of the invention, the therapeutically active agent is a cyclooxygenase (COX) inhibitor.

According to some embodiments of the invention, the therapeutically active agent is a non-steroidal anti-inflammatory drug (NTHE).

According to some embodiments of the invention, the therapeutically active agent is selected from the group consisting of salicyclic acid and mesalazine.

According to an aspect of some embodiments of the present invention, there is provided a chemical conjugate comprising a first moiety and a second moiety covalently linked therebetween, wherein the second moiety is derived from a fatty acid, and wherein the first moiety is derived from a food-grade or a derivative thereof each independently having a functional group for forming a covalent bond with the second moiety.

According to some embodiments of the invention, the chemical conjugate is further a 5-lipoxygenase (5-LOX) inhibitor.

According to some embodiments of the invention, the functional group is selected from the group consisting of hydroxy, amine, carboxy and amide.

According to some embodiments of the invention, the first moiety and the second moiety are covalently bound via a bond selected from the group consisting of an amide bond and an ester bond.

According to some embodiments of the invention, the fatty acid is docosa-4,7,10,13,16,19-hexaenoid acid.

According to some embodiments of the invention, the fatty acid is γ-linolenic acid.

According to some embodiments of the invention, the food additive is selected from the group consisting of quercetin, curcumin and resveratrol.

According to an aspect of some embodiments of the present invention there is provided a chemical conjugate comprising a first moiety and a second moiety covalently linked therebetween, wherein the first moiety is derived from a compound selected from the group consisting of 5-hydroxy-indol-3-yl-acetic acid, 2-amino-nicotinic acid, salicyclic acid, mesalazine, quercetin and resveratrol, and wherein the second moiety is derived from a fatty acid.

According to some embodiments of the invention, the first moiety and the second moiety are covalently linked therebetween via a bond selected from the group consisting of an ester bond and an amide bond.

According to some embodiments of the invention, the fatty acid is docosa-4,7,10,13,16,19-hexaenoic acid.

According to some embodiments of the invention, the fatty acid is y-linolenic acid.

According to some embodiments of the invention, the first moiety is derived from a compound selected from the group consisting of salicyclic acid and mesalazine.

According to an aspect of some embodiments of the present invention there is provided a chemical conjugate selected from the group consisting of:

N-(docosa-4,7,10,13,16,19-hexaenoyl)-5-hydroxy-indol-3-yl-aceticacid (MWL004);

N-(docosa-4,7,10,13,16,19-hexaenoyl)-2-amino-nicotinic acid (MWL005);

N-(docosa-4,7,10,13,16,19-hexaenoyl)-2-amino-phenyl-acetic acid (MWL006);

N-oleoyl-5-hydroxy-indol-3-yl-acetic acid (MWL007);

N-oleoyl-2-amino-nicotinic acid (MWL008);

O-oleoyl-salicylic acid (MWL009);

O-(docosa-4,7,10,13,16,19-hexaenoyl)-salicylic acid (MWL013);

O-(γ-linolenoyl)-salicylic acid (MWL014);

N-(γ-linolenoyl)-5-amino-salicylic acid (MWL015);

N-(docosa-4,7,10,13,16,19-hexaenoyl)-5-amino-salicylic acid (MWL016);

N-oleoyl-5-amino-salicylic acid (MWL017); and

N-docosa-4,7,10,13,16,19-hexaenoyl-taurine (MWL002).

According to an aspect of some embodiments of the present invention there is provided a N-docosa-4,7,10,13,16,19-hexaenoyl-taurine.

According to an aspect of some embodiments of the present invention there is provided a pharmaceutical composition comprising the chemical conjugate as described herein and a pharmaceutically acceptable carrier.

According to some embodiments of the invention, the pharmaceutical composition is packaged in a packaging material and identified in print, in or on the packaging material, for use in the treatment of an inflammatory disease or disorder.

According to an aspect of some embodiments of the present invention there is provided a chemical conjugate as described herein, for use in the treatment of an inflammatory disease or disorder.

According to an aspect of some embodiments of the present invention there is provided a use of the chemical conjugate as described herein in the manufacture of a medicament for the treatment of an inflammatory disease or disorder.

According to an aspect of some embodiments of the present invention there is provided a method of treating an inflammatory disease or disorder, the method comprising administering to a subject in need thereof an effective amount of the chemical conjugate as described herein.

According to some embodiments of the invention, the inflammatory disease or disorder is selected from the group consisting of Alzheimer's disease, Parkinson's disease, asthma, osteoarthritis, dermatitis, rheumatoid arthritis, pain associated with inflammation, primary dysmenorrhea, Crohn's disease and ulcerative colitis.

According to some embodiments of the invention, the conjugate inhibits COX-2 activity.

According to some embodiments of the invention, the conjugate further inhibits 5-LOX activity.

According to some embodiments of the invention, the conjugate does not inhibit COX-1 activity.

According to some embodiments of the invention, by conjugating DHA with specific amino acids, the compound's inhibition efficacy is significantly improved. The conjugates were built and synthesized to inhibit COX-2 in selective and reversible inhibition, with inhibition lasting only for a very short period. This unique mechanism of action exerts enough selective inhibition of COX-2 while maintaining an optimal COX-1/COX-2 ratio so that the body has the necessary levels of COX-2 enzyme to generate proper amounts of AA metabolites to maintain normal body functions.

According to some embodiments of the invention, the conjugate selectively inhibits COX-2 while maintaining an optimal balance of COX/LOX enzymes to maintain normal body functions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing the average COX-2 inhibition molecular activity index (MAI) of COX-2 of fatty acid amide derivatives formed by attaching exemplary amine-containing compounds to oleic acid (1), linoleic acid (2), a-linolenic acid (3), arachidonic acid (4), eicosa-5,8,11,14,17-pentaenoic acid (5) and docosa-4,7,10,13,16,19-hexaenoic acid (6).

FIGS. 2A-D present results from a representative oocyte expressing hERG channels before and after injection with MWL002 (15 μM). FIG. 2A presents currents before injection. FIG. 2B presents currents 5 minutes after injection. FIG. 2C presents voltage activation curves for the oocyte measured in FIGS. 2A and 2B. Presented are normalized currents at −130 mV that were initiated by the indicated voltage. FIG. 2D presents currents during 6-minute-long measurements prior and post injection of MLW002, as indicated by the gray bar. Currents were initiated by a 150-ms-long pulse to +40 and measured at −130 mV, with 15 seconds interpulse intervals.

FIG. 3 is a bar graph presenting average changes in expressed hERG currents following internal and external exposure to MWL002, and demonstrating no sensitivity of the channels to the tested conjugates.

FIG. 4 presents comparative plots showing the effect of MWL-001 and of ibuprofen on paw swelling volume in paw edema in vivo studies.

FIGS. 5A-B present the effect of MWL-001 and of 5-ASA on body weight (FIG. 5A) and on MPO activity (FIG. 5B) in UC-diseased rats.

FIGS. 6A-B present images of untreated (FIG. 6A) and of 5-ASA-treated (FIG. 6B, right) and MWL-001-treated (FIG. 6B, left) ulcerated colon segment.

FIGS. 7A-B are bar graphs showing the effect of MWL-001 on the PGE2 production (FIG. 7B) and the levels of TNFα (FIG. 7B) in CIA mice.

FIGS. 8A-B present comparative plots showing the arthritis score (FIG. 8A) and paw thickness (FIG. 8B) of ibuprofen-treated and MWL-002-treated CIA mice.

FIG. 9 presents the pharmacokinetic profile of MWL001 and DHA (it's metabolite) after oral administration in rats.

FIG. 10 presents the effect of MWL001 administered bolus I.V. on the rat QT and QTc intervals.

FIG. 11 presents the effect of quinidine (QND) on rat QT, QTc and heart rate.

FIG. 12 presents the TNF-α colon level of mice at four days after the administration of DNBS.

FIG. 13 presents the IL-6 colon level of mice at four days after the administration of DNBS.

FIG. 14 shows the representative immunolocalization of TGF-β expression in the colon tissues of mice on day four after the administration of DNBS.

FIG. 15 shows the representative immunolocalization of CD25 expression in the colon tissues of mice on day four after the administration of DNBS.

FIG. 16 shows the representative immunolocalization of CD4 expression in the colon tissues of mice on day four after the administration of DNBS.

FIG. 17 presents effect of MWL001 and dexamathasone (DEX) on ear thickness of mice at eighteen hours after sensitization.

FIG. 18 presents the ear weight of mice at eighteen hours after sensitization.

FIGS. 19 A-D representative hematoxylin/eosin-stained sections of mice ear tissues; A-control B-D sensitized with Oxazolone; B-with vehicle; C with MW001 and D with DEX.

### DESCRIPTION OF SPECIFIC EMBODIMENTS OF THE INVENTION

The present invention, in some embodiments thereof, relates to chemical conjugates and more particularly, to conjugates of a fatty acid and a therapeutically active agent, which can be used as COX-2 inhibitors for treating inflammation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, currently sought agents for treating inflammation are selective inhibitors of COX-2, which desirably do not inhibit COX-1. Dual inhibitors of COX and 5-LOX are also sought for obtaining a wider scope of anti-inflammatory activity.

The there-dimensional (3D) structure of the two enzymes, COX-1 and COX-2, determined by X-ray diffraction, shows that while the active site in both enzymes consists of a long narrow hydrophobic channel extending from the membrane-binding domain (the lobby) to the core of the catalytic domain, yet the COX 2 active site is about 20% larger and has a slightly different form as compared with that of COX-1.

These size and shape differences are caused mainly by two changes in the amino acid sequences of the isoenzymes. In one case, Ile-523 in COX 1 is replaced by a valine in COX-2, a change which opens up a small hydrophilic side pocket off the main channel; appreciably increasing the longer side chain of Ile-523. In addition, Ile-434 in COX-1 is also replaced by valine in COX-2, allowing a neighboring residue Phe-518 to swing out of the way, increasing further access to the side cavity. In another case, His-513 in COX-1, which can interact with polar moieties, is replaced by Arg in COX-2, thus changing the interaction of the side pocket with its chemical environment. The hydrophilic side pocket of the COX 2 active site is defined by residues Tyr-355, Val-523, His-90, Gln-192 and Arg-513.

The differences between COX-1 and COX-2 are further discussed in, for example, Dannhardt and, Kiefer, Eur J Med Chem. 2001 February; 36(2):109-26.

Currently, more than 500 COX-2-specific inhibitors have been designed. The main structural features of these compounds are the absence of the carboxylate group, characteristic of classical NSAIDs, and generally, the presence of a sulfonate (SO₂) or sulfonamide (SO-2NH2) moiety, which can interact with Arg-513 in the hydrophilic side pocket of the COX 2 active site. Although the majority of these compounds were discovered before the structure of COX-2 was dissolved, crystallographic data can now be used to rationally design selective inhibitors.

The present inventor has previously disclosed, in WO 08/075366, conjugates of various fatty acids and amine-containing compounds, and uses thereof in the treatment of inflammation.

Triggered by the knowledge obtained thus far on the active site of COX-2 and its structural differences from the active site of COX-1, the present inventor has now used in silico analyses in order to better define the structural features required for fatty acid-containing conjugates to selectively inhibit COX-2 and thereby serve as efficient anti-inflammatory agents.

The present inventors have focused on therapeutically active agents, mostly such agents that are naturally-occurring agents, as defined herein, and/or are determined as food-grade or Generally Recognized As Safe (GRAS) substances and even edible substance, as these defined herein, and have tested in silico the binding of conjugates of these agents with various fatty acids to COX-2. The chemical structures of exemplary substances are presented in Table 1 below. The fatty acids used in these studies were selected as exhibiting pharmacological benefits on their own, being of the family of Omega-3 fatty acids.

TABLE 1

| Compound | |
|---|---|
| No. | Compound Structure |
| 1 | 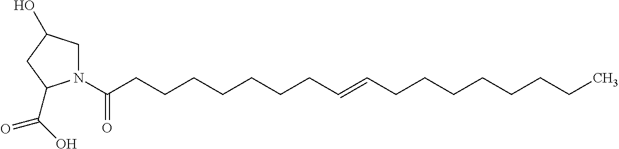 [SMILES: CCCCCCCC/C=C/CCCCCCCC(=O)N1CC(O)CC1C(=O)O] |
| 2 | 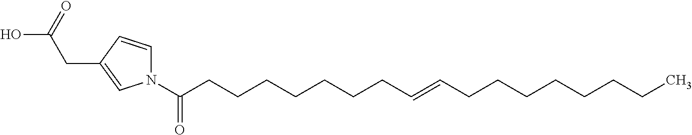 [SMILES: CCCCCCCC/C=C/CCCCCCCC(=O)n1ccc(CC(=O)O)c1] |
| 3 | 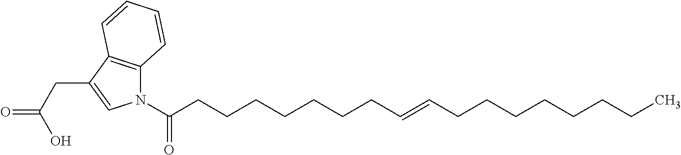 [SMILES: CCCCCCCC/C=C/CCCCCCCC(=O)n1cc(CC(=O)O)c2ccccc21] |
| 4 | 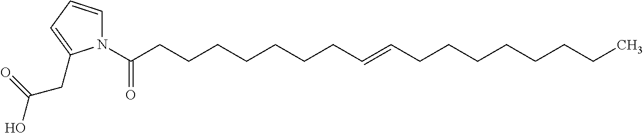 [SMILES: CCCCCCCC/C=C/CCCCCCCC(=O)n1cccc1CC(=O)O] |
| 5 | 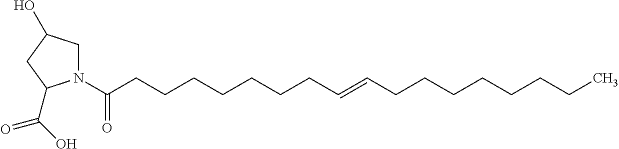 [SMILES: CCCCCCCC/C=C/CCCCCCCC(=O)N1CC(O)CC1C(=O)O] |
| 6 | 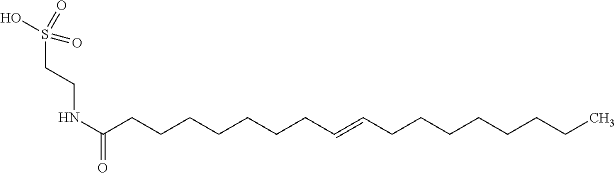 [SMILES: CCCCCCCC/C=C/CCCCCCCC(=O)NCCS(=O)(=O)O] |
| 7 | 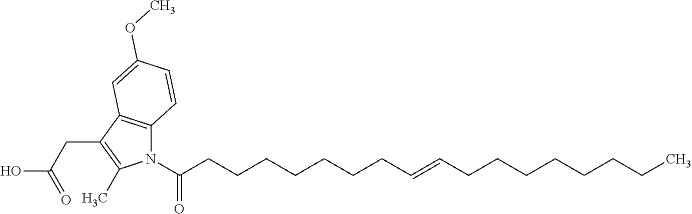 [SMILES: CCCCCCCC/C=C/CCCCCCCC(=O)n1c(C)c(CC(=O)O)c2cc(OC)ccc21] |
| 8 | 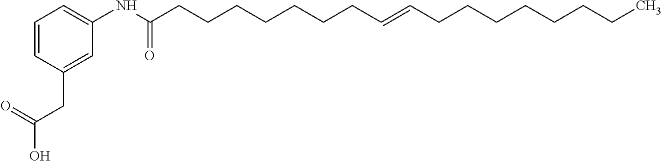 [SMILES: CCCCCCCC/C=C/CCCCCCCC(=O)Nc1cccc(CC(=O)O)c1] |
| 9 | 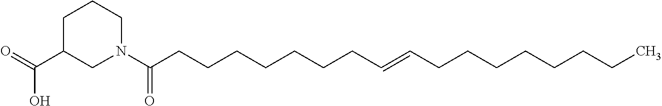 [SMILES: CCCCCCCC/C=C/CCCCCCCC(=O)N1CCCC(C(=O)O)C1] |
| 10 | 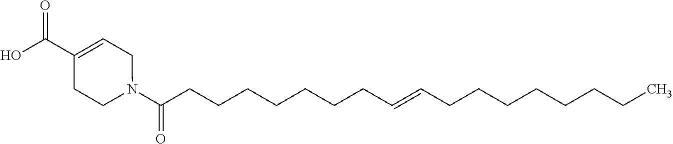 [SMILES: CCCCCCCC/C=C/CCCCCCCC(=O)N1CC=C(C(=O)O)CC1] |
| 11 | 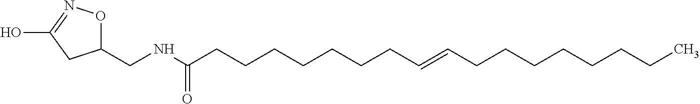 [SMILES: CCCCCCCC/C=C/CCCCCCCC(=O)NCC1CC(O)=NO1] |
| 12 | 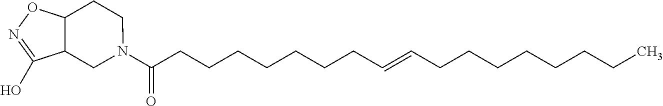 [SMILES: CCCCCCCC/C=C/CCCCCCCC(=O)N1CCC2ON=C(O)C2C1] |
| 13 | 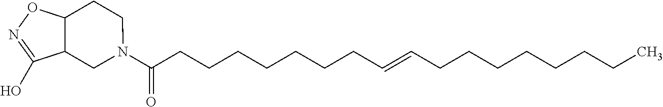 [SMILES: CCCCCCCC/C=C/CCCCCCCC(=O)N1CCC2ON=C(O)C2C1] |
| 14 | 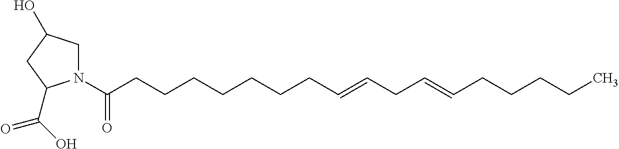 [SMILES: CCCCC/C=C/C/C=C/CCCCCCCC(=O)N1CC(O)CC1C(=O)O] |
| 15 | 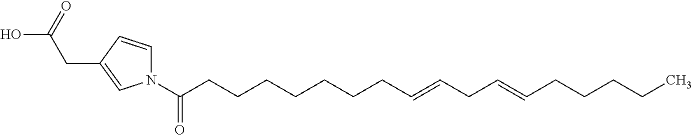 [SMILES: CCCCC/C=C/C/C=C/CCCCCCCC(=O)n1ccc(CC(=O)O)c1] |
| 16 | 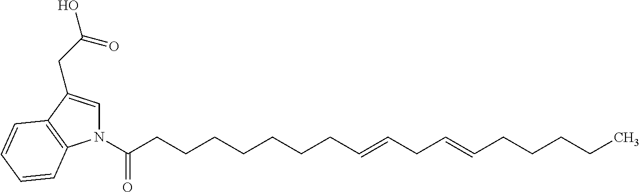 [SMILES: CCCCC/C=C/C/C=C/CCCCCCCC(=O)n1cc(CC(=O)O)c2ccccc21] |
| 17 | 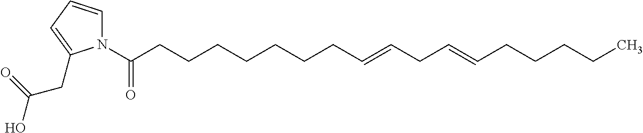 [SMILES: CCCCC/C=C/C/C=C/CCCCCCCC(=O)n1cccc1CC(=O)O] |
| 18 | 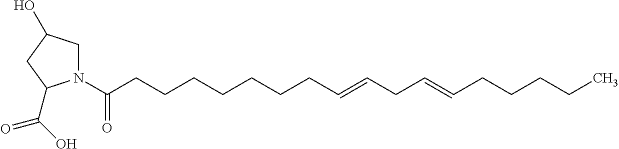 [SMILES: CCCCC/C=C/C/C=C/CCCCCCCC(=O)N1CC(O)CC1C(=O)O] |
| 19 | 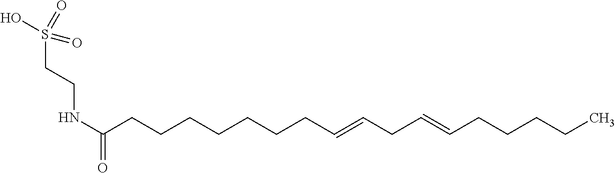 [SMILES: CCCCC/C=C/C/C=C/CCCCCCCC(=O)NCCS(=O)(=O)O] |
| 20 | 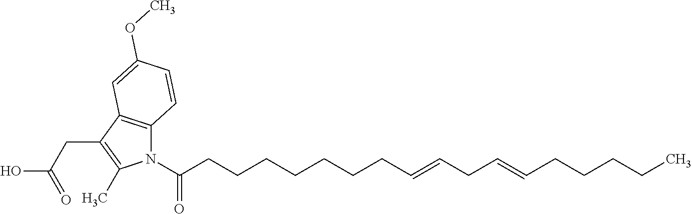 [SMILES: CCCCC/C=C/C/C=C/CCCCCCCC(=O)n1c(C)c(CC(=O)O)c2cc(OC)ccc21] |
| 21 | 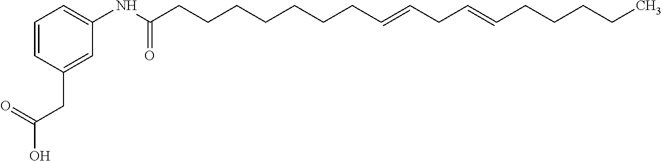 [SMILES: CCCCC/C=C/C/C=C/CCCCCCCC(=O)Nc1cccc(CC(=O)O)c1] |
| 22 | 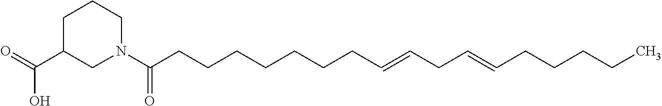 [SMILES: CCCCC/C=C/C/C=C/CCCCCCCC(=O)N1CCCC(C(=O)O)C1] |
| 23 | 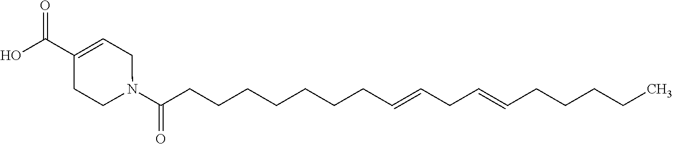 [SMILES: CCCCC/C=C/C/C=C/CCCCCCCC(=O)N1CC=C(C(=O)O)CC1] |
| 24 | 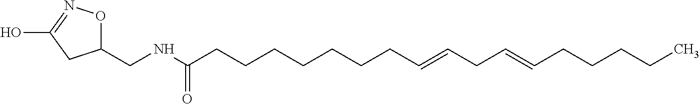 [SMILES: CCCCC/C=C/C/C=C/CCCCCCCC(=O)NCC1CC(O)=NO1] |
| 25 | 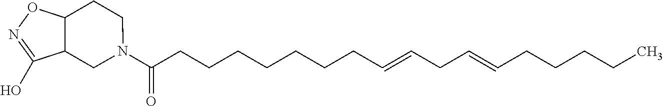 [SMILES: CCCCC/C=C/C/C=C/CCCCCCCC(=O)N1CCC2ON=C(O)C2C1] |
| 26 | 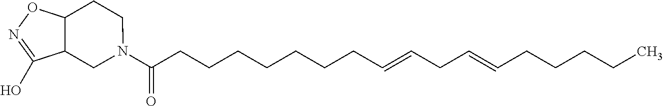 [SMILES: CCCCC/C=C/C/C=C/CCCCCCCC(=O)N1CCC2ON=C(O)C2C1] |
| 27 | 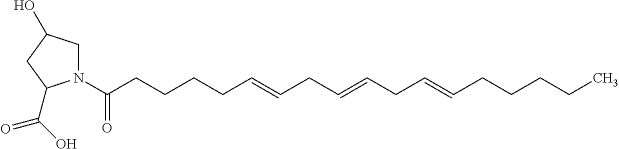 [SMILES: CCCCC/C=C/C/C=C/C/C=C/CCCCC(=O)N1CC(O)CC1C(=O)O] |
| 28 | 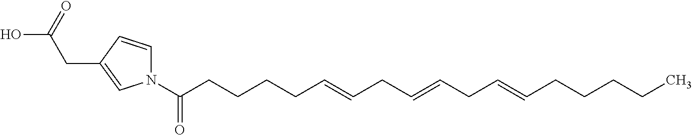 [SMILES: CCCCC/C=C/C/C=C/C/C=C/CCCCC(=O)n1ccc(CC(=O)O)c1] |
| 29 | 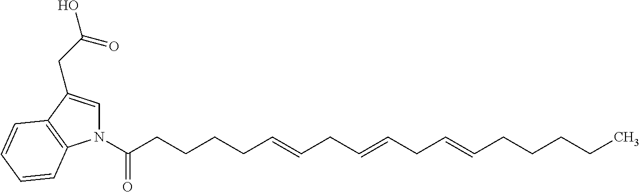 [SMILES: CCCCC/C=C/C/C=C/C/C=C/CCCCC(=O)n1cc(CC(=O)O)c2ccccc21] |
| 30 | 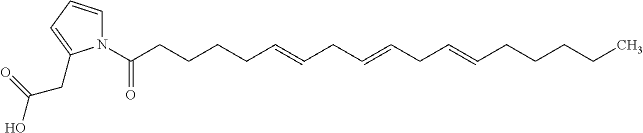 [SMILES: CCCCC/C=C/C/C=C/C/C=C/CCCCC(=O)n1cccc1CC(=O)O] |
| 31 | 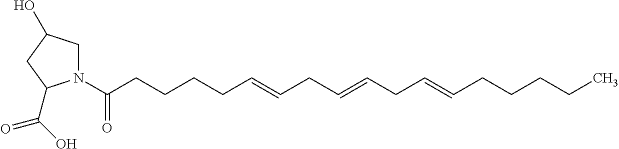 [SMILES: CCCCC/C=C/C/C=C/C/C=C/CCCCC(=O)N1CC(O)CC1C(=O)O] |
| 32 | 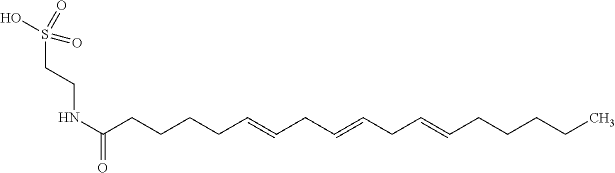 [SMILES: CCCCC/C=C/C/C=C/C/C=C/CCCCC(=O)NCCS(=O)(=O)O] |
| 33 | 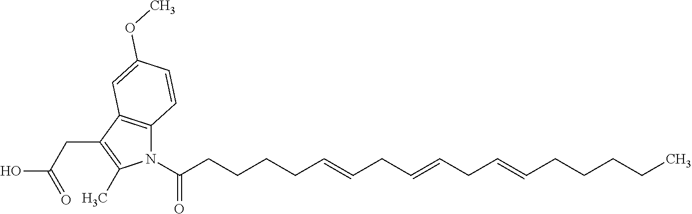 [SMILES: CCCCC/C=C/C/C=C/C/C=C/CCCCC(=O)n1c(C)c(CC(=O)O)c2cc(OC)ccc21] |
| 34 | 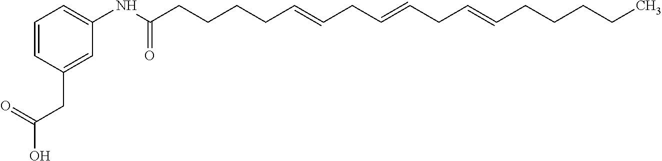 [SMILES: CCCCC/C=C/C/C=C/C/C=C/CCCCC(=O)Nc1cccc(CC(=O)O)c1] |
| 35 | 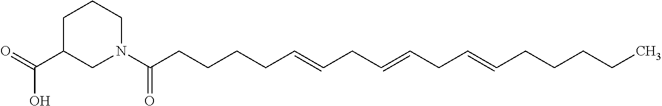 [SMILES: CCCCC/C=C/C/C=C/C/C=C/CCCCC(=O)N1CCCC(C(=O)O)C1] |
| 36 | 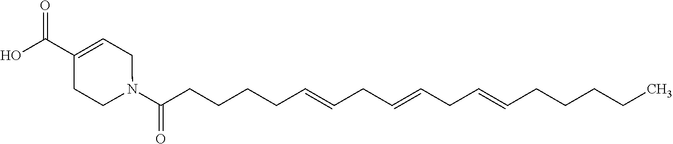 [SMILES: CCCCC/C=C/C/C=C/C/C=C/CCCCC(=O)N1CC=C(C(=O)O)CC1] |
| 37 | 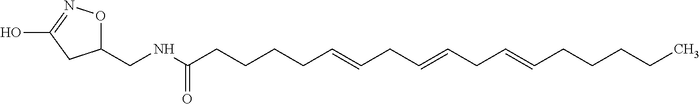 [SMILES: CCCCC/C=C/C/C=C/C/C=C/CCCCC(=O)NCC1CC(O)=NO1] |
| 38 | 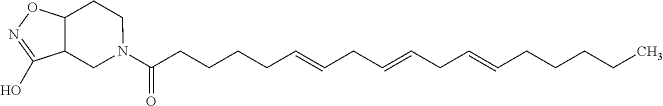 [SMILES: CCCCC/C=C/C/C=C/C/C=C/CCCCC(=O)N1CCC2ON=C(O)C2C1] |
| 39 | 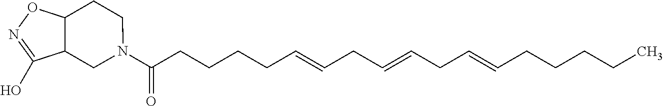 [SMILES: CCCCC/C=C/C/C=C/C/C=C/CCCCC(=O)N1CCC2ON=C(O)C2C1] |
| 40 | 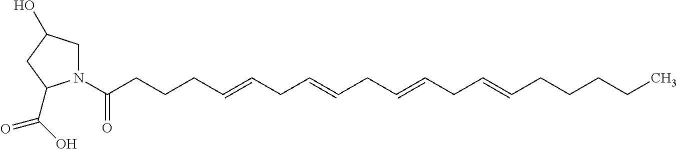 [SMILES: CCCCC/C=C/C/C=C/C/C=C/C/C=C/CCCC(=O)N1CC(O)CC1C(=O)O] |
| 41 | 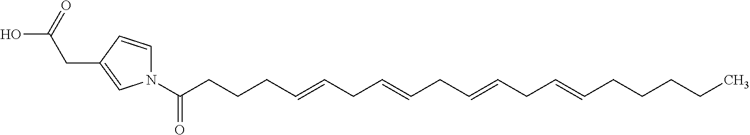 [SMILES: CCCCC/C=C/C/C=C/C/C=C/C/C=C/CCCC(=O)n1ccc(CC(=O)O)c1] |
| 42 | 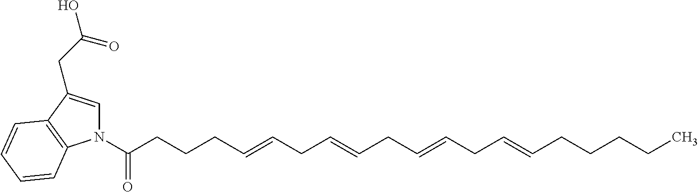 [SMILES: CCCCC/C=C/C/C=C/C/C=C/C/C=C/CCCC(=O)n1cc(CC(=O)O)c2ccccc21] |
| 43 | 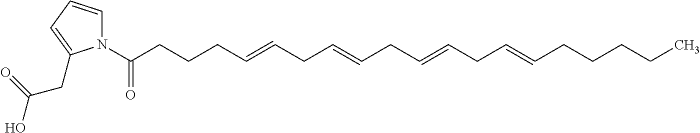 [SMILES: CCCCC/C=C/C/C=C/C/C=C/C/C=C/CCCC(=O)n1cccc1CC(=O)O] |
| 44 | 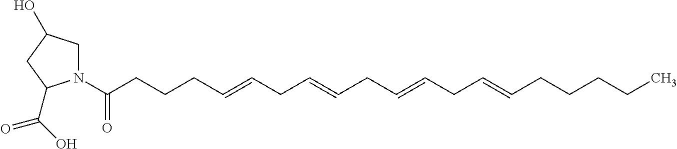 [SMILES: CCCCC/C=C/C/C=C/C/C=C/C/C=C/CCCC(=O)N1CC(O)CC1C(=O)O] |
| 45 | 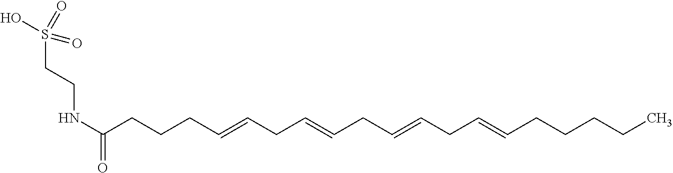 [SMILES: CCCCC/C=C/C/C=C/C/C=C/C/C=C/CCCC(=O)NCCS(=O)(=O)O] |
| 46 | 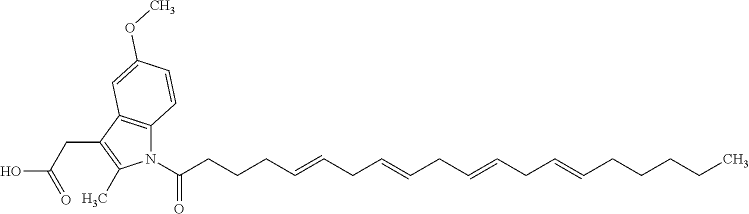 [SMILES: CCCCC/C=C/C/C=C/C/C=C/C/C=C/CCCC(=O)n1c(C)c(CC(=O)O)c2cc(OC)ccc21] |
| 47 | 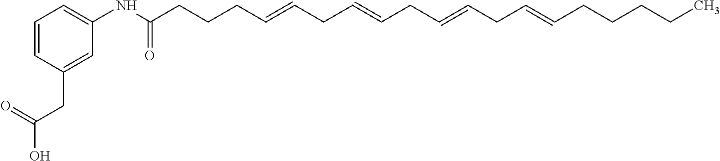 [SMILES: CCCCC/C=C/C/C=C/C/C=C/C/C=C/CCCC(=O)Nc1cccc(CC(=O)O)c1] |
| 48 | 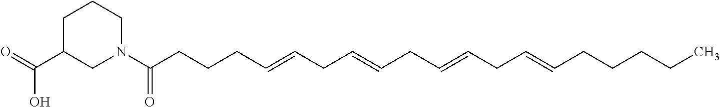 [SMILES: CCCCC/C=C/C/C=C/C/C=C/C/C=C/CCCC(=O)N1CCCC(C(=O)O)C1] |
| 49 | 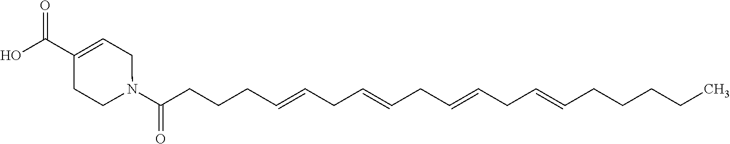 [SMILES: CCCCC/C=C/C/C=C/C/C=C/C/C=C/CCCC(=O)N1CC=C(C(=O)O)CC1] |
| 50 | 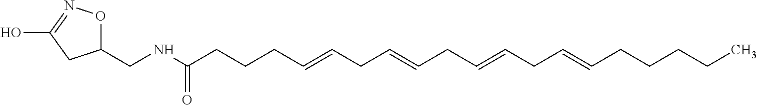 [SMILES: CCCCC/C=C/C/C=C/C/C=C/C/C=C/CCCC(=O)NCC1CC(O)=NO1] |
| 51 | 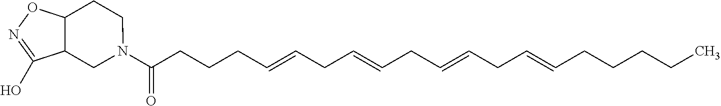 [SMILES: CCCCC/C=C/C/C=C/C/C=C/C/C=C/CCCC(=O)N1CCC2ON=C(O)C2C1] |
| 52 | 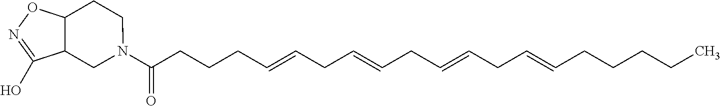 [SMILES: CCCCC/C=C/C/C=C/C/C=C/C/C=C/CCCC(=O)N1CCC2ON=C(O)C2C1] |
| 53 | 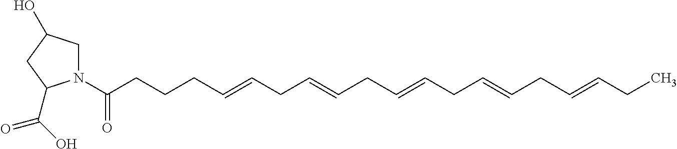 [SMILES: CC/C=C/C/C=C/C/C=C/C/C=C/C/C=C/CCCC(=O)N1CC(O)CC1C(=O)O] |
| 54 | 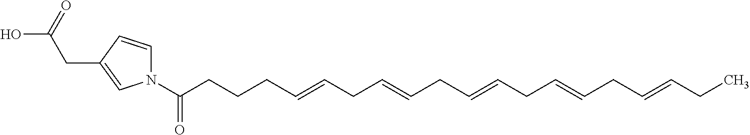 [SMILES: CC/C=C/C/C=C/C/C=C/C/C=C/C/C=C/CCCC(=O)n1ccc(CC(=O)O)c1] |
| 55 | 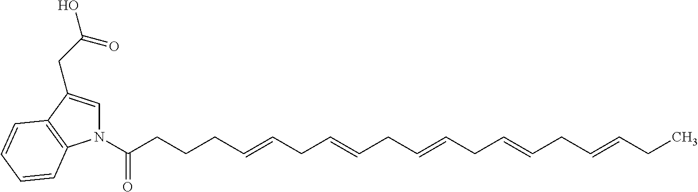 [SMILES: CC/C=C/C/C=C/C/C=C/C/C=C/C/C=C/CCCC(=O)n1cc(CC(=O)O)c2ccccc21] |
| 56 | 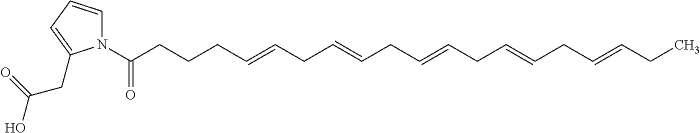 [SMILES: CC/C=C/C/C=C/C/C=C/C/C=C/C/C=C/CCCC(=O)n1cccc1CC(=O)O] |
| 57 | 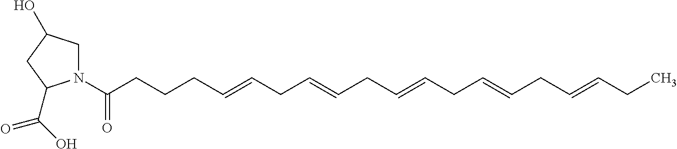 [SMILES: CC/C=C/C/C=C/C/C=C/C/C=C/C/C=C/CCCC(=O)N1CC(O)CC1C(=O)O] |
| 58 | 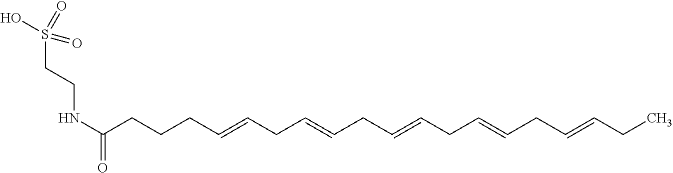 [SMILES: CC/C=C/C/C=C/C/C=C/C/C=C/C/C=C/CCCC(=O)NCCS(=O)(=O)O] |
| 59 | 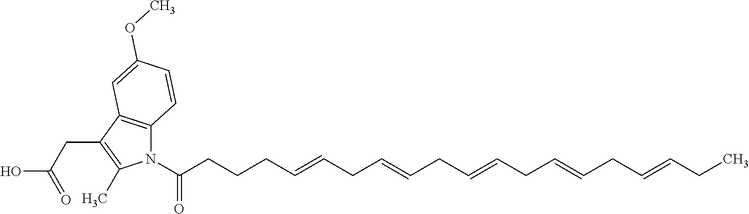 [SMILES: CC/C=C/C/C=C/C/C=C/C/C=C/C/C=C/CCCC(=O)n1c(C)c(CC(=O)O)c2cc(OC)ccc21] |
| 60 | 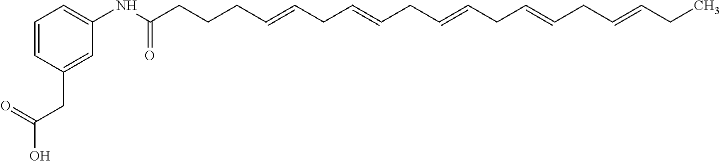 [SMILES: CC/C=C/C/C=C/C/C=C/C/C=C/C/C=C/CCCC(=O)Nc1cccc(CC(=O)O)c1] |
| 61 | 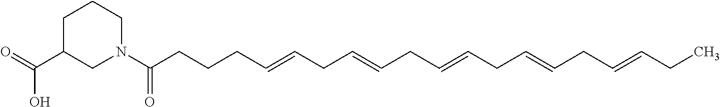 [SMILES: CC/C=C/C/C=C/C/C=C/C/C=C/C/C=C/CCCC(=O)N1CCCC(C(=O)O)C1] |
| 62 | 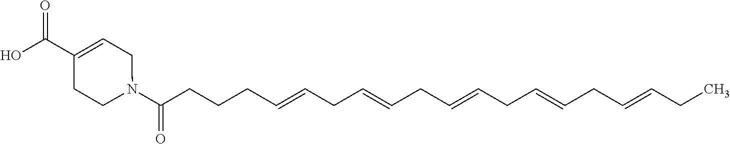 [SMILES: CC/C=C/C/C=C/C/C=C/C/C=C/C/C=C/CCCC(=O)N1CC=C(C(=O)O)CC1] |
| 63 | 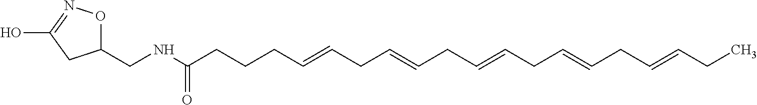 [SMILES: CC/C=C/C/C=C/C/C=C/C/C=C/C/C=C/CCCC(=O)NCC1CC(O)=NO1] |
| 64 | 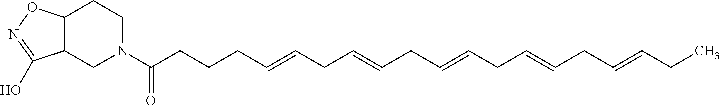 [SMILES: CC/C=C/C/C=C/C/C=C/C/C=C/C/C=C/CCCC(=O)N1CCC2ON=C(O)C2C1] |
| 65 | 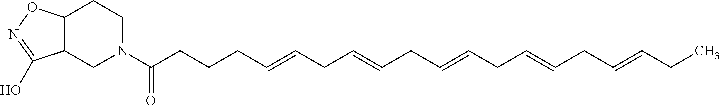 [SMILES: CC/C=C/C/C=C/C/C=C/C/C=C/C/C=C/CCCC(=O)N1CCC2ON=C(O)C2C1] |
| 66 | 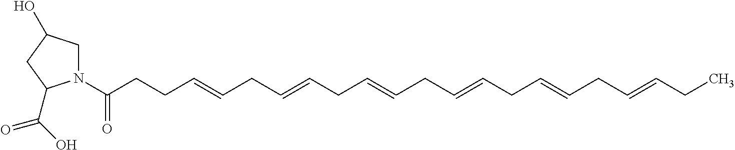 [SMILES: CC/C=C/C/C=C/C/C=C/C/C=C/C/C=C/C/C=C/CCC(=O)N1CC(O)CC1C(=O)O] |
| 67 | 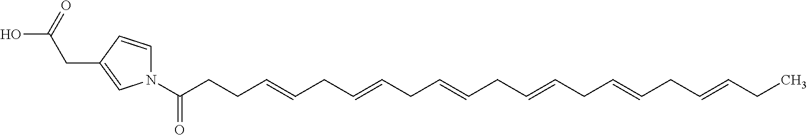 [SMILES: CC/C=C/C/C=C/C/C=C/C/C=C/C/C=C/C/C=C/CCC(=O)n1ccc(CC(=O)O)c1] |
| 68 | 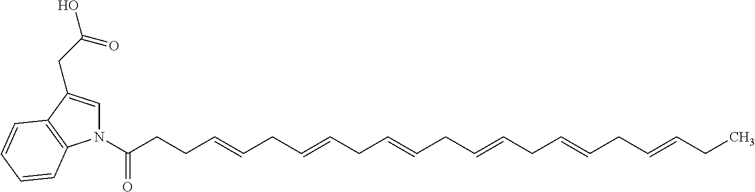 [SMILES: CC/C=C/C/C=C/C/C=C/C/C=C/C/C=C/C/C=C/CCC(=O)n1cc(CC(=O)O)c2ccccc21] |
| 69 | 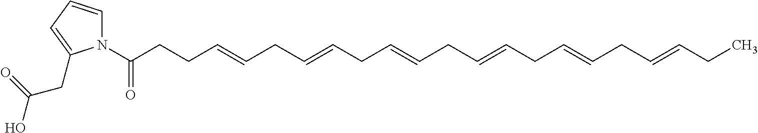 [SMILES: CC/C=C/C/C=C/C/C=C/C/C=C/C/C=C/C/C=C/CCC(=O)n1cccc1CC(=O)O] |
| 70 | 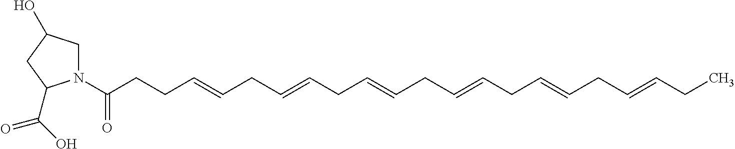 [SMILES: CC/C=C/C/C=C/C/C=C/C/C=C/C/C=C/C/C=C/CCC(=O)N1CC(O)CC1C(=O)O] |
| 71 | 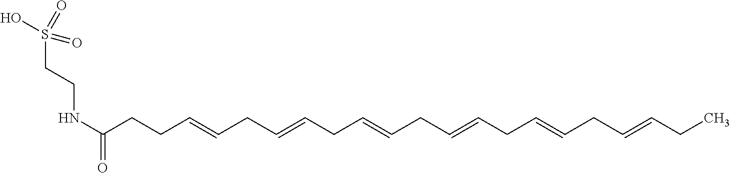 [SMILES: CC/C=C/C/C=C/C/C=C/C/C=C/C/C=C/C/C=C/CCC(=O)NCCS(=O)(=O)O] |
| 72 | 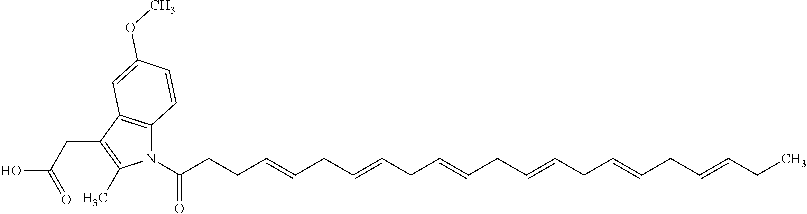 [SMILES: CC/C=C/C/C=C/C/C=C/C/C=C/C/C=C/C/C=C/CCC(=O)n1c(C)c(CC(=O)O)c2cc(OC)ccc21] |
| 73 | 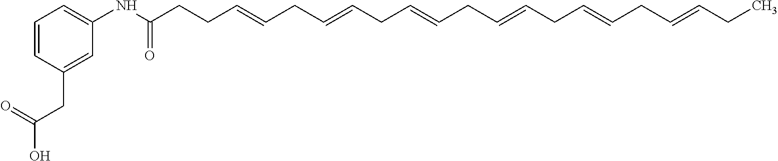 [SMILES: CC/C=C/C/C=C/C/C=C/C/C=C/C/C=C/C/C=C/CCC(=O)Nc1cccc(CC(=O)O)c1] |
| 74 | 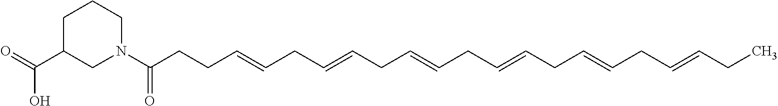 [SMILES: CC/C=C/C/C=C/C/C=C/C/C=C/C/C=C/C/C=C/CCC(=O)N1CCCC(C(=O)O)C1] |
| 75 | 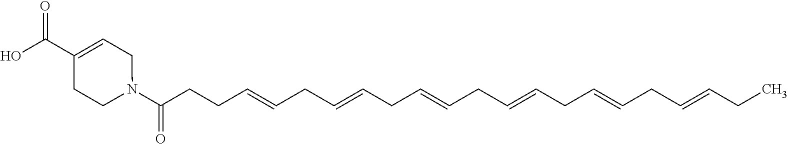 [SMILES: CC/C=C/C/C=C/C/C=C/C/C=C/C/C=C/C/C=C/CCC(=O)N1CC=C(C(=O)O)CC1] |
| 76 | 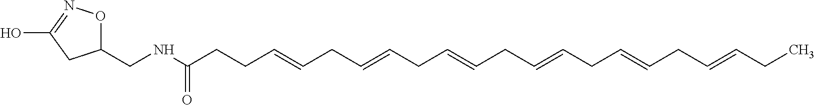 [SMILES: CC/C=C/C/C=C/C/C=C/C/C=C/C/C=C/C/C=C/CCC(=O)NCC1CC(O)=NO1] |
| 77 | 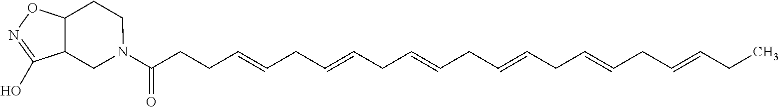 [SMILES: CC/C=C/C/C=C/C/C=C/C/C=C/C/C=C/C/C=C/CCC(=O)N1CCC2ON=C(O)C2C1] |
| 78 | 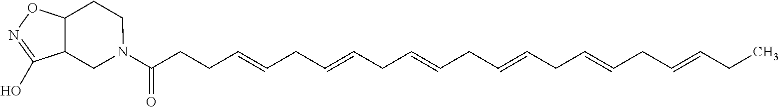 [SMILES: CC/C=C/C/C=C/C/C=C/C/C=C/C/C=C/C/C=C/CCC(=O)N1CCC2ON=C(O)C2C1] |

As described in detail in the Examples section that follows, the present inventors have uncovered that the degree of unsaturation in the fatty acid moiety has a substantial effect on the COX-2 binding of the studied conjugates, with conjugates comprised of a fatty acid having six double bonds exhibiting the best scores.

The pharmacological properties and therapeutic activity of exemplary compounds according to embodiments of the invention can be established in in vitro and in vivo studies, as further detailed hereinafter.

Embodiments of the present invention therefore generally relate to conjugates of a therapeutically active agent and a fatty acid.

More specifically, embodiments of the present invention relate to conjugates of a therapeutically active agent and a highly unsaturated fatty acid (e.g., DHA or linolenic acid), which are shown herein to have a superior therapeutic activity as compared to conjugates containing other fatty acids (e.g., mono-unsaturated or di-unsaturated fatty acids, having one or two double bonds, respectively). Further embodiments of the invention relate to conjugates of a therapeutically active agent and a fatty acid, both of which are derived from naturally-occurring substances, and hence can be categorized as food-grade or GRAS substances.

The chemical conjugates described herein are advantageously prepared by a one-step chemical synthesis, via a single bond conjugation. The chemical conjugates described herein exhibit high selectivity towards COX-2 inhibition, with IC₅₀ values being 200 to 500 folds lower than COX-1. Some of the tested conjugates advantageously exhibit a dual COX and 5-LOX inhibition activity.

According to one aspect of embodiments of the present invention there is provided a chemical conjugate comprising a first moiety and a second moiety covalently linked therebetween.

According to some embodiments of the present invention, the first moiety is derived from a therapeutically active agent or a derivative thereof, and the second moiety is derived from a fatty acid.

According to some embodiments of the present invention, the first moiety is an active agent or a derivative thereof, or a food additive or a derivative thereof and the second moiety is derived from a fatty acid.

By “derived from” it is meant that the moiety in the chemical conjugate is the portion of the substance forming the conjugate which remains upon the conjugation reaction with the other substance forming the conjugate. The phrase “derived from” further encompasses a portion of therapeutically active agent which possess most but not all of the structural features of the therapeutically active agent. For example, (5-hydroxy-1H-indol-3-yl)-acetic acid is a moiety derived from indomethacin.

As used herein throughout, the phrase “therapeutically active agent” relates to an agent that exhibits a beneficial pharmacological effect when administered to a subject. Exemplary therapeutically active agents include, but are not limited to, agents that exhibit anti-inflammatory activity, agents that exhibit anti-proliferative activity, anti-oxidants, anti-thrombogenic agents, anti-platelet agents, anti-coagulants, antimicrobial agents, analgesics, and vasoactive agents, as well as metabolites and biological substances such as, but not limited to, amino acids, nicotinic acid, and the like.

A derivative of a therapeutically active agent includes a substance, which has essentially the same structural features of the therapeutically active agent, yet is modified at one or more positions so as to possess a desired functional group. For example, a derivative of a therapeutically active agent can include an agent modified to include a hydroxy group, an amine group, a carboxy group and/or an amide group.

In some embodiments, the therapeutically active agent and the fatty acid are covalently linked therebetween via a bond formed between a functional group of the therapeutically active agent and the carboxylic group of the fatty acid.

The active agents may be therapeutically active agents or food-grade or food additive according to embodiments of the present invention therefore possess a functional group that serves for conjugating these agents to the fatty acid.

Suitable functional groups include an amine group, which forms an amide bond upon conjugation to a fatty acid, and a hydroxy group, which forms an ester bond upon conjugation to a fatty acid.

The hydroxy and amine functional groups can be present per se or may form a part of an amide or carboxy groups of the therapeutically active agent.

Accordingly, the therapeutically active agent and the fatty acid are linked therebetween via an amide bond or an ester bond. Corresponding moieties include a therapeutically active agent or a derivative thereof, possessing a —NR— group or a —O— group, as the first moiety, and a fatty acid possessing a —C(═O)— group, as the second moiety.

However, other functional groups and bonds formed thereby are contemplated. These include, but are not limited to, thiol, which forms upon conjugation with a fatty acid a thioester; carbamate, thiocarboxy, phosphonyl, phosphinyl, phosphoryl, phosphoramide, sulfate, sulfonate, sulfonamide, alkoxy, aryloxy, thioalkoxy, thioaryloxy, imine and halo.

It should be noted that the amine, hydroxy and thiol groups can be present in the therapeutically active agent either per se or can form a part of a ring, e.g., a heteroalicyclic or an heteroaromatic ring, or of a functional group such as amide, imine, ether, thioether, carboxy, thiocarboxy, carbamate, thiocarbamate and the like, as these terms are defined herein.

The functional group can be present in the therapeutically active agent or can be generated therein, so as to form a derivative of the therapeutically active agent.

In some embodiments, the active agent is an amino acid. Any of the currently known amino acids is contemplated, including the 21 naturally-occurring amino acids and non naturally-occurring amino acids, and any derivatives thereof.

In some embodiments, the therapeutically active agent is an anti-inflammatory agent. Exemplary anti-inflammatory agents include, but are not limited to, steroidal anti-inflammatory agents and non-steroidal anti-inflammatory agents.

In some embodiments, the therapeutically active agent is a non-steroidal anti-inflammatory agent.

Representative examples of non-steroidal anti-inflammatory agents include, without limitation, aspirin, celecoxib, diclofenac, diflunisal, etodolac, fenoprofen, flurbiprofen, ibuprofen, indomethacin, ketoprofen, ketorolac, meclofenamate, mefenamic acid, nabumetone, naproxen, oxaprozin, oxyphenbutazone, phenylbutazone, piroxicam, rofecoxib, sulindac and tolmetin.

More generally, examples of non-steroidal anti-inflammatory agents include, without limitation, oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene(fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof

Exemplary active agents from which the first moiety in the chemical conjugates described herein is derived include, but are not limited to, indomethacin, nicotinic acid (including derivatives thereof such as 2-amino nicotinic acid, 2-amino benzoic acid, and 2-aminophenyl acetic acid), salicyclic acid, mesalazine, quercetin, curcumin and resveratrol.

In some embodiments, the agent is a food-grade therapeutically active agent.

The phrase “food grade” or “food additive” is used herein to describe substances that are generally safe for human consumption by virtue of being generally recognized as safe (GRAS) or by passing standard safety tests, and thus qualify for use as food additives. This phrase describes those substances that are known to exhibit a therapeutic effect, either as nutritional supplements or as therapeutically active agents, as described herein.

The phrase “generally recognized as safe” or GRAS, as used herein, is meant in the same manner which is defined, for example, under sections 201(s) and 409 of the U.S. FD&C Act. The U.S. law states that any substance that intentionally contacts food or added to food is a food additive, that is subject to premarket review and approval by FDA, unless the substance is generally recognized, among qualified experts, as having been adequately shown to be safe under the conditions of its intended use, or unless the use of the substance is otherwise excluded from the definition of a food additive. GRAS substances are distinguished from food additives by the type of information that supports the GRAS determination, that it is publicly available and generally accepted by the scientific community, but should be the same quantity and quality of information that would support the safety of a food additive.

Since the qualification to a food additive (food-grade) or GRAS category can be obtained through a process of applying, testing and qualifying to the requirements of the various official food and drug authorities, the present embodiments are meant to encompass all relevant substances and their derivatives which are to become food-grade and GRAS in the future, as well as those which already qualify as food-grade and GRAS.

In some embodiments, the therapeutically active agent is a naturally-occurring substance.

By “naturally-occurring” it is meant that the substance is found in natural plants or animals. Naturally-occurring substances can be obtained by extracting the substance from the plant or animal it is found in, or can be synthetically prepared.

The second moiety in the chemical conjugates described herein is derived from a fatty acid.

As commonly used in the art, a fatty acid is comprised of a hydrocarbon chain which terminates with a carboxylic acid group. The hydrocarbon chain can be unbranched and saturated, branched and saturated, unbranched and unsaturated or branched and unsaturated.

In some embodiment, the fatty acid is an unsaturated fatty acid having one or more unsaturated bonds (e.g., double bonds) in its hydrocarbon chain.

In some embodiments, the hydrocarbon chain is unbranched.

In some embodiments, the hydrocarbon chain comprises from 5 to 29 carbon atoms, rendering the fatty acid being of 6 to 30 carbon atoms in length.

In some embodiments, the fatty acid is of 16 to 22 carbon atoms in length.

In some embodiments, the fatty acid has at least 3 double bonds in its hydrocarbon chain. In some embodiments, the fatty acid has at least 4 double bonds in its hydrocarbon chain. In some embodiments, the fatty acid has at least 5 double bonds in its hydrocarbon chain. In some embodiments of the invention, the fatty acid has at least 6 double bonds in its hydrocarbon chain. The configuration of the double bonds in the hydrocarbon chain, namely cis or trans, can be the same or different.

In some embodiments, the unsaturated fatty acid is an all-cis unsaturated fatty acid.

In some embodiments, the fatty acid is an omega-3-fatty acid, as this term is widely recognized in the art.

Exemplary fatty acids that are advantageously used in the context of embodiments of the present invention include, but are not limited to, all-cis-7,10,13-hexadecatrienoic acid, all-cis-9,12,15-octadecatrienoic acid (α-Linolenic acid (ALA)), all-cis-6,9,12,15-octadecatetraenoic acid (Stearidonic acid (SDA)), all-cis-11,14,17-eicosatrienoic acid (Eicosatrienoic acid (ETE)), all-cis-8,11,14,17-eicosatetraenoic acid (Eicosatetraenoic acid (ETA)), all-cis-5,8,11,14,17-eicosapentaenoic acid (Eicosapentaenoic acid (EPA)), all-cis-7,10,13,16,19-docosapentaenoic acid (Docosapentaenoic acid (DPA), Clupanodonic acid), all-cis-4,7,10,13,16,19-docosahexaenoic acid (Docosahexaenoic acid (DHA), all-cis-9,12,15,18,21 -tetracosapentaenoic acid (Tetracosapentaenoic acid) and all-cis-6,9,12,15,18,21-tetracosahexaenoic acid (Tetracosahexaenoic acid (Nisinic acid)).

In some embodiments, the fatty acid is all-cis-4,7,10,13,16,19-docosahexaenoic acid (Docosahexaenoic acid (DHA).

In some embodiments, the fatty acid is linolenic acid.

According to some embodiments of the present invention, the first moiety is derived from a therapeutically active agent or a derivative thereof, as described herein, whereas hydroxyproline is excluded and the second moiety is derived from docosa-4,7,10,13,16,19-hexaenoic acid (DHA).

According to some embodiments of the present invention, the first moiety is derived from a therapeutically active agent or a derivative thereof, whereas hydroxyproline or taurine are excluded as described herein, and the second moiety is derived from linolenic acid.

According to some embodiments of the present invention, the first moiety is derived from a food-grade therapeutically active agent, as defined herein, and the second moiety is derived from a fatty acid, as defined herein.

According to some embodiments of the present invention, the first moiety is derived from 5-hydroxy-indol-3-yl-acetic acid, 2-amino-nicotinic acid, salicyclic acid, mesalazine, quercetin or resveratrol, or from any derivative thereof, and the second moiety is derived from a fatty acid.

In some embodiments, the first moiety is derived from 5-hydroxy-indol-3-yl-acetic acid (e.g., derived from indomethacin), including derivatives thereof as exemplified in Table 1 and in Example 1 that follows.

Exemplary compounds according to some embodiments of the present invention include, but are not limited to:

N-(docosa-4,7,10,13,16,19-hexaenoyl)-5-hydroxy-indol-3-yl-acetic acid (MWL004), and derivatives thereof;

N-(docosa-4,7,10,13,16,19-hexaenoyl)-2-amino-nicotinic acid (MWL005);

N-(docosa-4,7,10,13,16,19-hexaenoyl)-2-amino-phenyl-acetic acid (MWL006);

N-oleoyl-5-hydroxy-indol-3-yl-acetic acid (MWL007);

N-oleoyl-2-amino-nicotinic acid (MWL008);

O-oleoyl-salicylic acid (MWL009);

O-(docosa-4,7,10,13,16,19-hexaenoyl)-salicylic acid (MWL013);

O-(y-linolenoyl)-salicylic acid (MWL014);

N-(7-linolenoyl)-5-amino-salicylic acid (MWL015);

N-(docosa-4,7,10,13,16,19-hexaenoyl)-5-amino-salicylic acid (MWL016);

N-oleoyl-5-amino-salicylic acid (MWL017); and

N-docosa-4,7,10,13,16,19-hexaenoyl-taurine (MWL002).

The chemical conjugates described herein can be in a form of a pharmaceutically acceptable salt, a prodrug, a solvate or a hydrate thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

As used herein, the term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo. An example, without limitation, of a prodrug would be the chemical conjugate, having one or more carboxylic acid moieties, which is administered as an ester (the “prodrug”). Such a prodrug is hydrolysed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the NO-donating compound) and a solvent, whereby the solvent does not interfere with the biological activity of the solute.

Suitable solvents include, for example, ethanol, acetic acid and the like.

The term “hydrate” refers to a solvate, as defined hereinabove, where the solvent is water.

The term “alkyl”, as used herein, describes a saturated aliphatic hydrocarbon including straight chain and branched chain groups. In some embodiments, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. In some embodiments, the alkyl is a lower alkyl having 1 to 3 carbon atoms. The alkyl group may be substituted or unsubstituted, as indicated herein.

The term alkenyl, as used herein, describes an alkyl, as defined herein, which contains a carbon-to-carbon double bond.

The term alkynyl, as used herein, describes an alkyl, as defined herein, which contains carbon-to-carbon triple bond.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, alkyl, cycloalkyl, alkoxy, aryl and aryloxy, carbonyl, aldehyde and carboxy, depending on the substituted group and its position in the molecule.

The term “halide” or “halo” describes fluorine, chlorine, bromine or iodine.

The term “haloalkyl” describes an alkyl group as defined herein, further substituted by one or more halide.

The term “S-sulfonamide” describes a —S(═O)₂—NR′R″ group, with R′ as defined herein and R″ being as defined herein for R′.

The term “N-sulfonamide” describes an R′S(═O)₂—NR″— group, where R′ and R″ are as defined herein.

The terms “S-sulfonamide” and “N-sulfonamide” are collectively referred to herein as sulfonamide.

The term “thiocarbonyl” as used herein, describes a —C(═S)—R′ group, with R′ as defined herein.

The term “carbonyl” as used herein, describes a —C(═O)—R′ group, with R′ as defined herein.

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “sulfoxide” describes a —S(═O)R′ group with R′ being hydrogen, alkyl, cycloalkyl or aryl, as defined herein.

The term “phosphonate” describes a —P(═O)(OR′)(OR″) group, with R′ and R″ as defined herein.

The term “sulfonate” describes a —S(═O)₂—R′ group, where R′ is as defined herein.

The term “acyl halide” describes a —(C═O)R″″ group wherein R″″ is halide, as defined hereinabove.

The term “C-thiocarboxylate” describes a —C(═S)—OR′ group, where R′ is as defined herein.

The term “C-carboxylate” describes a —C(═O)—OR′ group, where R′ is as defined herein.

The term “O-thiocarboxylate” describes a —OC(═S)R′ group, where R′ is as defined herein.

The term “N-carbamate” describes an R″OC(═O)—NR′— group, with R′ and R″ as defined herein.

The term “O-carbamate” describes an —OC(═O)—NR′R″ group, with R′ and R″ as defined herein.

The term “O-thiocarbamate” describes a —OC(═S)—NR′R″ group, with R′ and R″ as defined herein.

The term “N-thiocarbamate” describes an R″OC(═S)NR′— group, with R′ and R″ as defined herein.

The term “S-dithiocarbamate” describes a —SC(═S)—NR′R″ group, with R′ and R″ as defined herein.

The term “N-dithiocarbamate” describes an R″SC(═S)NR′— group, with R′ and R″ as defined herein.

The term “C-amide” describes a —C(═O)—NR′R″ group, where R′ and R″ are as defined herein.

The term “N-amide” describes a R′C(═O)—NR″— group, where R′ and R″ are as defined herein.

The terms “N-amide” and “C-amide” are collectively referred to herein as amide.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

As discussed hereinabove, the chemical conjugates as described herein, were designed and practiced so as to selectively inhibit COX-2.

Accordingly, in some embodiments, the chemical conjugates described herein are identified as COX-2 inhibitors.

The phrase “COX-2 inhibitor” describes a compound (e.g., a chemical conjugate as described herein) which is capable of substantially inhibiting an activity of COX-2, whereby the phrase “selective COX-2 inhibitor” describes a compound has an inhibitory activity towards COX-2 which is substantially higher than its inhibitory activity towards COX-1.

In some embodiments, the chemical conjugates described herein are characterized as an inhibitory activity towards COX-2 which is higher by at least 100-folds than its inhibitory activity towards COX-1.

In some embodiments, the chemical conjugates described herein are characterized by an inhibitory activity towards COX-2 which is 100-folds, 200 folds, 300-folds, 400-folds, 500-folds or 1000-folds or more than the inhibitory activity towards COX-1.

Methods for determining an inhibitory activity of a compound towards COX-1 and COX-2 are well known in the art. Exemplary methods are described in the Examples section that follows.

In some embodiments, the chemical conjugates described herein are characterized by an inhibitory activity towards 5-LOX.

In some embodiments, the chemical conjugates described herein are advantageously characterized by a dual effect of inhibiting both COX (e.g., COX-2) and 5-LOX. Methods for determining an inhibitory activity of a compound towards 5-LOX are well known in the art.

Data demonstrating such an inhibition activity has been obtained for exemplary conjugates as described herein, yet is not shown herein.

As noted hereinabove, inhibition of the 5-Lipoxygenase (5-LOX) pathway reduces the production of Leukotriene B4 (LTB4), a potent chemoattractant molecule of white blood cells which can cause additional inflammation at the site of injury. Elevated LTB4 has been shown to contribute to gastric damage in mucosal lesions. Accordingly, a dual inhibition effect of COX and 5-LOX reduces AA metabolites, but allows the body to maintain pools of these necessary AA metabolites to perform essential functions.

As further noted hereinabove, agents exhibiting a dual inhibition effect of COX and 5-LOX are highly potent in treating a wider spectrum of inflammatory conditions.

Accordingly, according to an aspect of embodiments of the present invention, the chemical conjugates described herein are identified for use in the treatment of an inflammatory disease or disorder.

According to an aspect of embodiments of the invention there is provided a method of treating an inflammatory disease or disorder, which is effected by administering to a subject in need thereof a therapeutically effective amount of a chemical conjugate as described herein.

According to an aspect of embodiments of the present invention there is provided a use of any of the chemical conjugates described herein as a medicament.

In some embodiments, the medicament is for treating an inflammatory disease or disorder.

Exemplary inflammatory disease or disorder that are treatable by the chemical conjugates described herein include, but are not limited to, Alzheimer's disease, cortical dementia, vascular dementia, muli-infract dementia, pre-senile dementia, alcoholic dementia, senile dementia, memory loss or central nervous damage resulting from stroke, ischemia or trauma, multiple sclerosis, Parkinson's disease, Huntington's disease, epilepsy, cystic fibrosis, arthritis diseases such as osteoarthritis, rheumatoid arthritis, spondyloarthopathies, gouty arthritis, systemic lupus erythematosus, and juvenile arthritis fever, periarteritis; gastrointestinal disorders such as inflammatory bowel disease, Chron's disease, gastritis, irritable bowel syndrome, ulcerative colitis, cardiovascular disorders such as myocardial ischemia, reperfusion injury to an ischemic organ; angiogenesis, asthma, bronchitis, menstrual cramps, premature labor, tendinitis, bursitis, an autoimmune disease, an immunological disorder, systemic lupus erythematosus, inflammatory disorders of the skin such as psoriasis, eczema, burns and dermatitis; neoplasia, an inflammatory process in a disease, pulmonary inflammation, a central nervous system disorder, migraine headaches, allergic rhinitis, respiratory distress syndrome, endotoxin shock syndrome, a microbial infection, a bacterial-induced inflammation, a viral induced inflammation, a urinary disorder, a urological disorder, endothelial dysfunction, organ deterioration, tissue deterioration, adhesion and infiltration of neutrophils at the site of inflammation, thyroiditis, aplastic anemia, Hodgkin's disease, sclerodoma, rheumatic fever, myasthenia gravis, sarcoidosis, nephrotic syndrome, Behcet's syndrome, polymyositis, hypersensitivity, conjunctivitis, gingivitis and swelling occurring after injury.

In some embodiments, the inflammatory disease or disorder that are treatable by the chemical conjugates described herein include, but are not limited to, Alzheimer's disease, Parkinson's disease, asthma, osteoarthritis, dermatitis, rheumatoid arthritis, pain associated with inflammation, primary dysmenorrhea, Crohn's disease and ulcerative colitis.

The chemical conjugates described herein can be administered via local administration or systemically, e.g., orally, rectally, intravenously, intraventricularly, topically, intranasally, intraperitoneally, intestinally, parenterally, intraocularly, intradermally, transdermally, subcutaneously, intramuscularly, transmucosally, by inhalation and/or by intrathecal catheter. In some embodiments of the invention, the chemical conjugates described herein are administered orally or intravenously, and optionally topically, transdermally or by inhalation, depending on the condition and the subject being treated.

In some embodiments, the inflammatory disease or disorder that are treatable by the chemical conjugates described herein is a skin or mucosal disease or disorder, or is manifested by skin or mucosal ailments. In these embodiments, the chemical conjugate can be administered topically and accordingly is formulated for topical application, as detailed hereinbelow.

In any of the methods and uses described herein, the chemical conjugate can be utilized either per se or being formulated into a pharmaceutical composition which further comprises a pharmaceutically acceptable carrier.

Hence, according to still another aspect of the present invention, there are provided pharmaceutical compositions, which comprise one or more of the chemical conjugates described above and a pharmaceutically acceptable carrier.

As used herein a “pharmaceutical composition” refers to a preparation of one or more of the chemical conjugates described herein, with other chemical components such as pharmaceutically acceptable and suitable carriers and excipients. The purpose of a pharmaceutical composition is to facilitate administration of a compound to an organism.

Hereinafter, the phrase “pharmaceutically acceptable carrier” describes a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the agents described herein into preparations which, can be used pharmaceutically. Proper formulation is dependent upon the route of administration chosen.

According to some embodiments, the pharmaceutical composition is formulated as a solution, suspension, emulsion or gel.

According to some embodiments, the pharmaceutical composition further includes a formulating agent selected from the group consisting of a suspending agent, a stabilizing agent and a dispersing agent.

For injection, the agents described herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

For oral administration, the agents described herein can be formulated readily by combining the chemical conjugates with pharmaceutically acceptable carriers well known in the art. Such carriers enable the agents described herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate. Also oral compositions may comprise at least one flavorant such as, but not limited to, wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, and cinnamon leaf oil.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active agent doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the agent(s) may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents described herein are conveniently delivered in the form of an aerosol spray presentation (which typically includes powdered, liquified and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the chemical conjugate and a suitable powder base such as, but not limited to, lactose or starch.

The agents described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the agents described herein in water-soluble form. Additionally, suspensions of the agents may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the agents to allow for the preparation of highly concentrated solutions.

Alternatively, the chemical conjugates may be in powder form for constitution with a suitable vehicle, e.g., sterile, pyrogen-free water, before use.

The chemical conjugates described herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

By selecting the appropriate carrier and optionally other ingredients that can be included in the composition, as is detailed hereinbelow, the compositions of the present invention may be formulated into any form typically employed for topical application. Hence, the compositions of the present invention can be, for example, in a form of a cream, an ointment, a paste, a gel, a lotion, a milk, a suspension, an aerosol, a spray, a foam, a shampoo, a hair conditioner, a serum, a swab, a pledget, a pad, a patch and a soap.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emolliency). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-α-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir, patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition.

Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions.

Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Pharmaceutical compositions for topical application as described herein can be identified also as cosmetic or cosmeceutic products.

Pharmaceutical compositions suitable for use in context of the present invention include compositions wherein the active ingredients are contained in an amount effective to achieve the intended purpose. More specifically, a therapeutically effective amount means an amount of a chemical conjugate as described herein effective to prevent, alleviate or ameliorate symptoms of a physiological disorder associated with oxidative stress (such as tobacco-associated damage) or prolong the survival of the subject being treated.

Determination of a therapeutically effective amount is well within the capability of those skilled in the art, especially in light of the detailed disclosure provided herein.

For any chemical conjugate or an additional agent utilized in the methods and uses of the invention, the therapeutically effective amount or dose can be estimated initially from activity assays in animals. For example, a dose can be formulated in animal models to achieve a circulating concentration range that includes the IC₅₀ as determined by activity assays (e.g., the concentration of the test agent, which achieves a half-maximal reduction in cell death upon exposure to cigarette smoke). Such information can be used to more accurately determine useful doses in humans.

Toxicity and therapeutic efficacy of the agents described herein can be determined by standard pharmaceutical procedures in experimental animals, e.g., by determining the EC₅₀, the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present embodiments may, if desired, be presented in a pack or dispenser device, such as an FDA (the U.S. Food and Drug Administration) approved kit, which may contain one or more unit dosage forms containing the active agent. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising an agent as described herein, formulated in a compatible pharmaceutical carrier may also be prepared, placed in an appropriate container, and labeled for treatment of an indicated condition, as is detailed herein.

Thus, according to an embodiment of the present invention, the pharmaceutical composition is packaged in a packaging material and identified in print, in or on the packaging material, for use in the treatment of an inflammatory disease or disorder, as described herein.

In any of the compositions, methods and uses described herein, the chemical conjugates can be utilized in combination with an additional therapeutically active agent. In some embodiments, the additional therapeutically active agent is an anti-inflammatory agent. In some embodiments the additional active agent is a COX-2 inhibitor and/or a 5-LOX inhibitor.

It is to be noted that some of the compounds encompassed by embodiments of the invention have been described in the art. Such compounds are excluded from the scope of embodiments of the invention that relate to chemical conjugates per se, yet are included in embodiments of the invention that relate to the use of such conjugates as exhibiting beneficial anti-inflammatory activity.

As used herein the term “about” refers to ±10%.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### In Silico Studies

The COX-2-inhibition ability of the compounds set forth in Table 1 containing a fatty acid moiety attached to an amine moiety via an amide bond was determined in silico, in order to determine the effect of the type of a fatty acid and the type of a therapeutically active agent containing an amine functional group (hereinafter also referred to as “an amine-containing compound”) on COX-2 inhibition.

### Experimental Methods

A set of conjugates deriving from all possible combinations of 6 fatty acids and 13 amine-containing compounds, as depicted in Table 2 below, attached to one another via an amide bond was screened using procedures analogous to those described, for example, in WO 2009/090613 and in Falah et al., Bioinformation 3(9):389-393 (2009). The fatty acids, amine-containing compounds, and the conjugates derived therefrom, are shown in Table 2.

The chemical structures of the chemical conjugates are depicted in Table 1 in the specification.

TABLE 2
Compound Nos. for exemplary compounds containing
an amine moiety attached to a fatty acid moiety

| Fatty acid moiety | | | | | | |
|---|---|---|---|---|---|---|
| Docosa- | Eicosa- | | | | | |
| 4,7,10,13,16,19- | 5,8,11,14,17- | | α- | | | |
| hexaenoic acid | pentaenoic acid | Arachidonic | Linolenic | Linoleic | Oleic | |
| (DHA) | (EPA) | acid | acid | acid | acid | Amine moiety |
| 66, 70 | 53,57 | 40, 44 | 27, 31 | 14, 18 | 1, 5 | hydroxyproline |
| 67 | 54 | 41 | 28 | 15 | 2 | pyrrol-3-yl-acetic |
| | | | | | | acid |
| 68 | 55 | 42 | 29 | 16 | 3 | indol-3-yl-acetic |
| | | | | | | acid |
| 69 | 56 | 43 | 30 | 17 | 4 | pyrrol-2-yl-acetic |
| | | | | | | acid |
| 71 | 58 | 45 | 32 | 19 | 6 | taurine |
| 72 | 59 | 46 | 33 | 20 | 7 | 5-methoxy-2- |
| | | | | | | methyl-indol-3- |
| | | | | | | yl-acetic acid |
| 73 | 60 | 47 | 34 | 21 | 8 | 3-amino-phenyl- |
| | | | | | | acetic acid |
| 74 | 61 | 48 | 35 | 22 | 9 | nipecotic acid |
| 75 | 62 | 49 | 36 | 23 | 10 | isoguvacine |
| 76 | 63 | 50 | 37 | 24 | 11 | 5-aminomethyl- |
| | | | | | | 4,5-dihydro- |
| | | | | | | isoxazol-3-ol |
| 77 | 64 | 51 | 38 | 25 | 12 | 3a,4,5,6,7,7a- |
| | | | | | | hexahydro- |
| | | | | | | isoxazolo[4,5- |
| | | | | | | c]pyridin-3-ol |
| 78 | 65 | 52 | 39 | 26 | 13 | 4,5,6,7- |
| | | | | | | tetrahydro- |
| | | | | | | isoxazolo[4,5- |
| | | | | | | c]pyridin-3-ol |

### Results

The molecular activity index (MAI) of each conjugate was calculated in a COX-2 inhibition model, as detailed hereinabove. The MAI values of the abovementioned conjugates set forth in Table 1 are summarized in Table 3 below, with the conjugates being ranked by magnitude of the MAI value. MAI values above zero were considered to be zero.

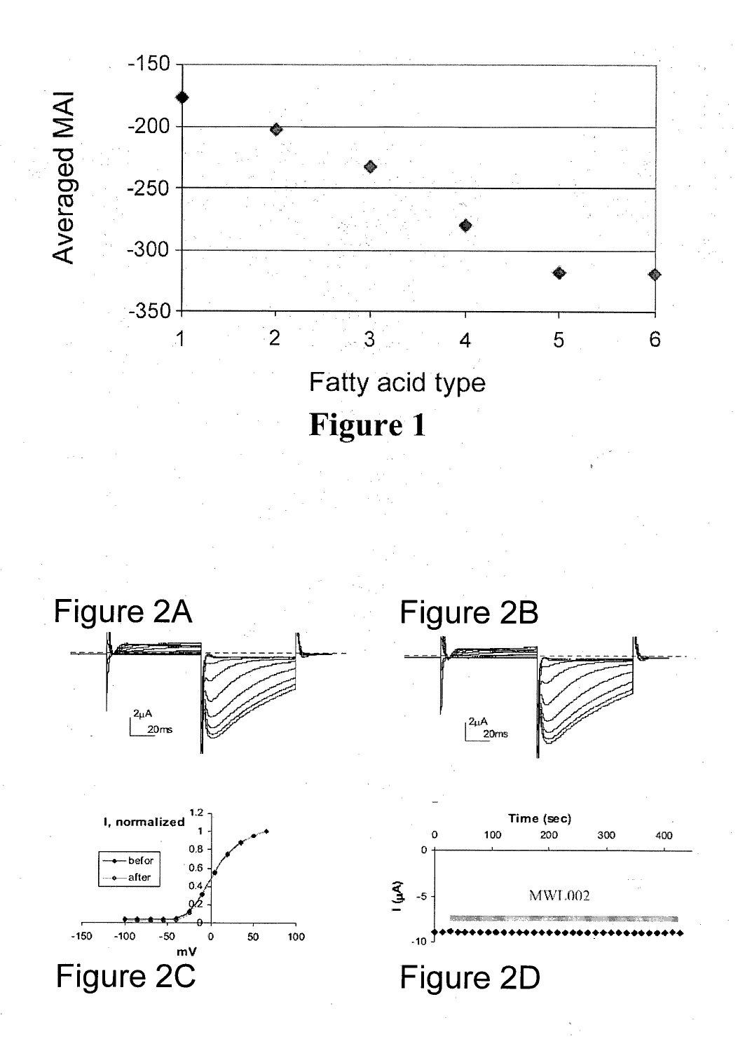

MAI values for the 13 conjugates associated with each fatty acid were averaged to obtain an average MAI value for each of the 6 fatty acids. The results are presented in FIG. 1.

As shown in FIG. 1, DHA exhibited the average MAI with the highest magnitude, followed closely by EPA. As further shown therein, the magnitude of the average MAI values of the fatty acids was correlated to the number of double bonds in the fatty acid.

These results indicate that DHA is particularly suitable for forming COX-2 inhibitors.

As shown in Table 3 below, analysis of the COX-2 inhibition activity correlation to the amine-containing compounds tested, reveals that 4 of the 10 compounds having the largest MAI values (i.e., compounds 65, 78, 52 and 39 in Tables 1 and 2) comprised a 4,5,6,7-tetrahydro-isoxazolo[4,5-c]pyridin-3-ol moiety, 3 of the 10 compounds having the largest MAI values (i.e., compounds 64, 51 and 77) and 4 of the top 11 compounds (i.e., compounds 64, 51, 77 and 38) comprised a 3a,4,5,6,7,7a-hexahydroisoxazolo[4,5-c]pyridin-3-ol moiety, and 2 of the 10 compounds having the largest MAI values (i.e., compounds 55 and 68) and 6 of the top 17 compounds (i.e., compounds 55, 68, 16, 29, 3 and 42) comprised an indol-3-yl-acetic acid moiety.

As further shown in Table 3 below, of the 25 compounds with the largest MAI values, four compounds comprised a 5-methoxy-2-methyl-indol-3-yl-acetic acid moiety, and five compounds comprised a 3-amino-phenyl-acetic acid moiety.

These results indicate that certain amine-containing moieties are particularly suitable for forming COX-2 inhibitors.

TABLE 3
Molecular activity index for compounds
of FIG. 1 (ranked by magnitude)

| Molecular Activity | |
|---|---|
| Index (MAI) | Compound No. |
| −654.0 | 55 |
| −601.6 | 65 |
| −591.3 | 78 |
| −581.0 | 52 |
| −576.5 | 64 |
| −570.6 | 51 |
| −558.0 | 77 |
| −545.1 | 68 |
| −526.9 | 39 |
| −512.0 | 59 |
| −510.7 | 38 |
| −427.0 | 16 |
| −427.0 | 29 |
| −395.5 | 3 |
| −389.1 | 72 |
| −360.1 | 33 |
| −358.3 | 42 |
| −352.2 | 47 |
| −329.1 | 21 |
| −328.2 | 60 |
| −325.8 | 34 |
| −324.9 | 20 |
| −304.2 | 67 |
| −299.3 | 76 |
| −296.9 | 8 |
| −291.1 | 43 |
| −290.8 | 69 |
| −263.3 | 74 |
| −261.5 | 75 |
| −257.3 | 41 |
| −253.6 | 54 |
| −247.8 | 62 |
| −247.8 | 26 |
| −245.5 | 49 |
| −244.2 | 56 |
| −242.0 | 73 |
| −235.1 | 7 |
| −229.5 | 48 |
| −219.7 | 61 |
| −210.6 | 46 |
| −204.9 | 66 |
| −204.9 | 70 |
| −201.0 | 25 |
| −188.5 | 40 |
| −188.5 | 44 |
| −186.7 | 13 |
| −178.2 | 23 |
| −176.4 | 53 |
| −176.4 | 57 |
| −171.1 | 17 |
| −170.7 | 50 |
| −167.7 | 15 |
| −162.7 | 27 |
| −162.7 | 31 |
| −160.9 | 22 |
| −160.3 | 12 |
| −156.5 | 10 |
| −152.4 | 2 |
| −152.2 | 1 |
| −152.2 | 5 |
| −150.5 | 4 |
| −146.6 | 14 |
| −146.6 | 18 |
| −140.2 | 63 |
| −135.3 | 30 |
| −132.6 | 9 |
| −127.3 | 24 |
| −124.8 | 35 |
| −121.0 | 11 |
| −114.6 | 28 |
| −108.8 | 36 |
| −64.3 | 37 |
| 0 | 71 |
| 0 | 58 |
| 0 | 45 |
| 0 | 6 |
| 0 | 19 |
| 0 | 32 |

### Example 2

Following the In Silico data presented in Example 1 hereinabove, exemplary conjugates according to embodiments of the invention were designed and successfully synthesized.

Materials and Methods:

Solvents and reagents were obtained from commercial suppliers and were used without further purification.

¹H NMR spectra were recorded in DMSO-D₆ or CDCl₃ on a Bruker WM300 spectrometer. Chemical shifts are given in p.p.m. relative to tetramethylsilane (¹H).

Thin layer chromatography (TLC) was performed on Merck Silica Gel 60 F₂₅₄ plates.

Column chromatography was performed using Merck silica gel 60.

General Procedures:

A general synthetic pathway for preparing conjugates of fatty acids and therapeutically active agents linked therebetween via an amide bond, according to some embodiments of the present invention, involves a condensation reaction of a fatty acid and an amine-containing compound.

A general synthetic pathway for preparing conjugates of fatty acids and therapeutically active agents linked therebetween via an ester bond, according to some embodiments of the present invention, involves a condensation reaction of a fatty acid and an alcohol-containing compound which does not contain an amine group.

Thus, according to a representative synthetic pathway, a desired conjugate is typically prepared, according to embodiments of the invention, by placing a corresponding fatty acid in a dry solvent such as dichloromethane or tetrahydrofuran, and adding dimethylaminopyridine (DMAP) and N,N-dicyclohexylcarbodiimide (DCC). The mixture is stirred at 0° C. for about 20 minutes, and a corresponding amine-containing compound or alcohol-containing compound is then added at an amount equimolar to the amount of fatty acid, and stirred at ambient temperature for about 20 hours. The solid residue is removed by filtration and the solvent is removed by evaporation. The desired conjugate is purified by being dissolved in a solvent such as n-hexane, removing the undissolved solid, and removing the solvent by evaporation.

Using the general procedure described above, a variety of conjugates according to embodiments of the present invention were prepared, as is detailed hereinbelow.

### Synthesis of N-docosa-4,7,10,13,16,19-hexaenoyl-5-hydroxy-indol-3-yl-acetic acid (MWL004)

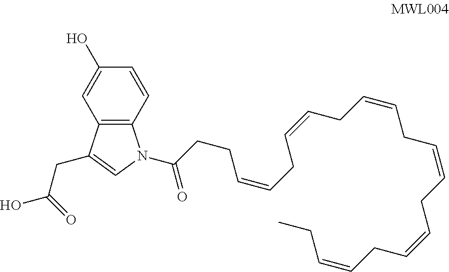 [SMILES: CC/C=C\C/C=C\C/C=C\C/C=C\C/C=C\C/C=C\CCC(=O)n1cc(CC(=O)O)c2cc(O)ccc21]

1 gram (3.04 mmol) of all-cis-DHA (all-cis-docosa-4,7,10,13,16,19-hexaenoic acid) was dissolved in 50 ml dry dichloromethane under an inert atmosphere, and 0.48 gram (3.39 mmol) dimethylaminopyridine (DMAP) and 0.88 gram (4.26 mmol) N,N′-dicyclohexylcarbodiimide (DCC) were then added. The mixture was stirred at 0° C. for 20 minutes, and 0.58 gram (3.04 mmol) of 5-hydroxy-indol-3-yl-acetic acid was added and stirred at ambient temperature for 20 hours. The solid residue was removed by filtration and the dichloromethane was removed by evaporation. The solid-oil mixture was dissolved in n-hexane, the undissolved solid was removed, and the hexane was then removed by evaporation. 2.33 grams of N-docosa-4,7,10,13,16,19-hexaenoyl-5-hydroxy-indol-3-yl-acetic acid was obtained as a dark brown solid.

¹H-NMR (CDCl₃): 1.02(t, 3H, CH₃), 2.09(q, 2H, CH₂CH₃), 2.29(m, 2H, CH₂CH₂—CH), 2.47(t,_{—}2H, CH₂CH₂—CH) 2.69(m, 10H, CH₂), 3.59(s, 2H, CH₂COOH), 5.37-3.58(m, 13H, All cis, and OH-Aromatic), 6.50(d, 1H, Aromatic), 7.39(s, 1H), 7.46(s, 1H, Aromatic), 7.60(d, 1H, Aromatic).

### Synthesis of N-(docosa-4,7,10,13,16,19-hexaenoyl)-2-amino-nicotinic acid (MWL005)

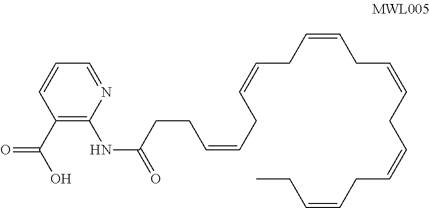 [SMILES: CC/C=C\C/C=C\C/C=C\C/C=C\C/C=C\C/C=C\CCC(=O)Nc1ncccc1C(=O)O]

N-(docosa-4,7,10,13,16,19-hexaenoyl)-2-amino-nicotinic acid (MWL005) was synthesized according to the general procedures described hereinabove, wherein the fatty acid was all-cis-DHA and the amine-containing compound was 2-amino-nicotinic acid.

¹H-NMR (CDCl₃): 1.07(t, 3H, CH₃), 2.00(q, 2H, CH₂CH₃), 2.23-2.51(m, 4H, CH₂CH₂), 2.79(m, 10H, CH₂), 5.35(m, 12H, All-cis), 6.86-7.25(m, 3H, Aromatic), 9.47(1H, NH—CO).

### Synthesis of N-(docosa-4,7,10,13,16,19-hexaenoyl)-2-amino-phenyl-acetic acid (MWL006)

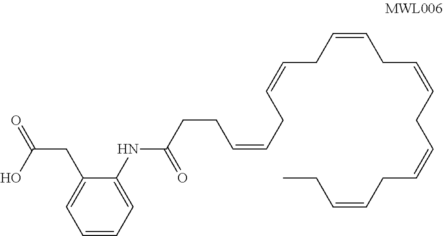 [SMILES: CC/C=C\C/C=C\C/C=C\C/C=C\C/C=C\C/C=C\CCC(=O)Nc1ccccc1CC(=O)O]

N-(docosa-4,7,10,13,16,19-hexaenoyl)-2-amino-phenyl-acetic acid (MWL006) was synthesized according to the general procedures described hereinabove, wherein the fatty acid was all-cis-DHA and the amine-containing compound was 2-amino-phenyl-acetic acid.

To a solution of 20 ml dry THF, 0.5 gram (1.52 mmol) DHA, and 0.45 ml triethylamine 0.144 ml of triethylchloroformate were added. A white solid immediately precipitated. The solution was stirred for another 15 minutes at room temperature, and the white solid was thereafter filtered out. The THF solution was then added to 230 mg of a 2-amino-phenyl-acetic acid in THF. The clear solution was then stirred at room temperature for additional 16 hours. The THF was removed and the resulting yellow powder was taken in dichloromethane and washed twice with water, dried over sodium sulfate and evaporated to dryness to afford 350 mg of a pale yellow solid (Yield: 49%).

¹H-NMR(CDCl₃): 1.1(t, 3H, CH₃), 2.05(q, 2H, CH₂CH₃), 2.23-2.51(m, 4H, CH₂CH₂), 2.69(m, 10H, CH₂), 3.72(s, 2H, CH₂—COOH), 5.42(m, 12H, All-cis), 7.01-7.32(m, 4H, Aromatic).

### Synthesis of 3′,4′,5,7-tetrahydroxy-flavone-3-yl docosa-4,7,10,13,16,19-hexaenoate (MWL011)

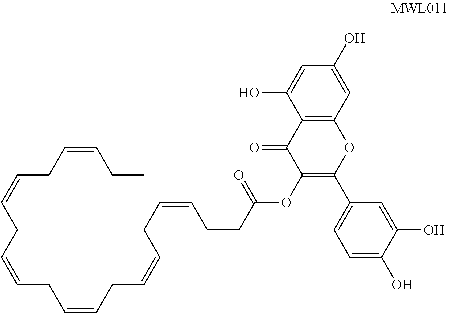 [SMILES: CC/C=C\C/C=C\C/C=C\C/C=C\C/C=C\C/C=C\CCC(=O)Oc1c(-c2ccc(O)c(O)c2)oc2cc(O)cc(O)c2c1=O]

3′,4′,5,7-tetrahydroxy-flavone-3-yl docosa-4,7,10,13,16,19-hexaenoate (MWL011) is synthesized according to the general procedures described hereinabove, wherein the fatty acid is DHA and the alcohol-containing compound is 3′,4′,3,5,7-pentahydroxy-flavone (quercetin).

### Synthesis of O-(docosa-4,7,10,13,16,19-hexaenoyl)-salicylic acid (MWL013)

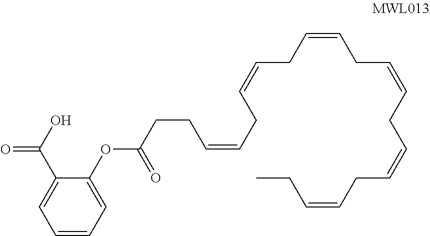 [SMILES: CC/C=C\C/C=C\C/C=C\C/C=C\C/C=C\C/C=C\CCC(=O)Oc1ccccc1C(=O)O]

O-(docosa-4,7,10,13,16,19-hexaenoyl)-salicylic acid (MWL013) was synthesized according to the general procedures described hereinabove, wherein the fatty acid is DHA and the alcohol-containing compound is salicylic acid.

To a solution of 20 ml dry THF, 0.5 gram (1.52 mmol) DHA, and 0.45 ml triethylamine, 0.144 ml triethylchloroformate were added. A white solid immediately participated. The solution was stirred for additional 15 minutes at room temperature, followed by filtration of the white solid. The THF solution was then added to 210 mg salicylic acid in THF. The clear solution was then stirred at room temperature for additional 22 hours. The THF was thereafter removed and the resulting solid was taken in dichloromethane and washed twice with water, dried over sodium sulfate and evaporated to dryness to afford 420 mg (0.93 mmol) of a white solid (yield: 62%).

¹H-NMR (CDCl₃): 1.02 (t, 3H, CH₃), 2.01 (q, 2H, CH₂CH₃), 2.23-2.51 (m, 4H, CH₂CH₂), 2.70 (m, 10H, CH₂), 5.35 (m, 12H, All-cis), 7.75-8.25 (m, 4H, Aromatic).

### Synthesis of N-(docosa-4,7,10,13,16,19-hexaenoyl)-5-amino-salicylic acid (MWL016)

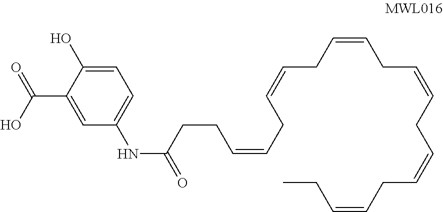 [SMILES: CC/C=C\C/C=C\C/C=C\C/C=C\C/C=C\C/C=C\CCC(=O)Nc1ccc(O)c(C(=O)O)c1]

N-(docosa-4,7,10,13,16,19-hexaenoyl)-5-amino-salicylic acid (MWL016) was synthesized according to the general procedures described hereinabove, wherein the fatty acid is DHA and the amine-containing compound is 5-amino-salicylic acid (mesalazine).

¹H-NMR(CDCl₃): 1.03 (t, 3H, CH₃), 2.00 (q, 2H, CH₂CH₃), 2.23-2.51 (m, 4H, CH₂CH₂), 2.74 (m, 10H, CH₂), 5.39 (s, 1H, OH-Aromatic), 5.39 (m, 12H, All-cis), 7.25 (d, 1H, Aromatic), 7.79 (1H, Aromatic), 8.30 (s, 1H, Aromatic).

### Synthesis of N-docosa-4,7,10,13,16,19-hexaenoyl-pyrrol-3-yl-acetic acid (MWL066)

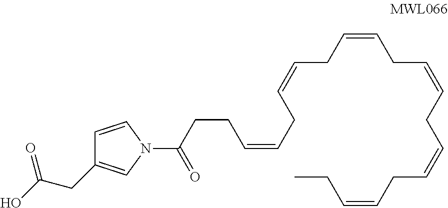 [SMILES: CC/C=C\C/C=C\C/C=C\C/C=C\C/C=C\C/C=C\CCC(=O)n1ccc(CC(=O)O)c1]

N-docosa-4,7,10,13,16,19-hexaenoyl-pyrrol-3-yl-acetic acid (MWL066) was synthesized according to the general procedures described hereinabove, wherein the fatty acid is DHA and the amine-containing compound is pyrrol-3-yl-acetic acid.

¹H-NMR (CDCl₃): 0.99 (t, 3H, CH₃), 2.00 (q, 2H, CH₂CH₃), 2.29 (q, 2H, CH₂CH₂), 2.31 (q, 2H, CH₂—CH₂), 2.64 (m, 10H, CH), 3.55 (s, 1H, CH₂—COOH), 5.40 (m, 12H, all cis), 6.00 (d, 1H, Aromatic), 6.79 (1H, Aromatic), 7.00 (d, 1H, Aromatic).

### Synthesis of N-docosa-4,7,10,13,16,19-hexaenoyl-indol-3-yl-acetic acid (MWL068)

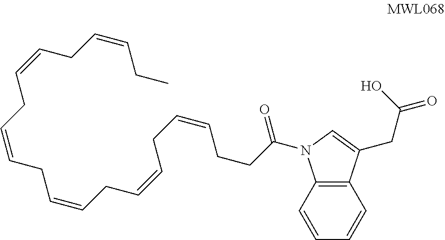 [SMILES: CC/C=C\C/C=C\C/C=C\C/C=C\C/C=C\C/C=C\CCC(=O)n1cc(CC(=O)O)c2ccccc21]

N-docosa-4,7,10,13,16,19-hexaenoyl-indol-3-yl-acetic acid (MWL068) was synthesized according to the general procedures described hereinabove, wherein the fatty acid is DHA and the amine-containing compound is indol-3-yl-acetic acid.

¹H-NMR(CDCl₃): 1.02 (t, 3H, CH₃), 2.09 (q, 2H, CH₂CH₃), 2.29 (m, 2H, CH₂CH₂—CH), 2.47 (t,_{—}2H, CH₂CH₂—CH) 2.69 (m, 10H, CH₂), 3.59 (s, 2H, CH₂COOH), 5.37-3.58 (m, 12H, All cis), 6.50 (m, 1H, Aromatic), 7.39 (s, 1H), 7.46 (m, 1H, Aromatic), 7.90 (m, 1H, Aromatic).

### Synthesis of N-docosa-4,7,10,13,16,19-hexaenoyl-5-methoxy-2-methyl-indol-3-yl-acetic acid (MWL072)

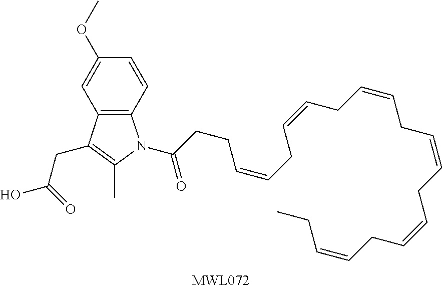 [SMILES: CC/C=C\C/C=C\C/C=C\C/C=C\C/C=C\C/C=C\CCC(=O)n1c(C)c(CC(=O)O)c2cc(OC)ccc21]

N-docosa-4,7,10,13,16,19-hexaenoyl-5-methoxy-2-methyl-indol-3-yl-acetic acid (MWL072) was synthesized according to the general procedures described hereinabove, wherein the fatty acid is DHA and the amine-containing compound is 5-methoxy-2-methyl-indol-3-yl-acetic acid.

¹H-NMR (CDCl₃): 1.07 (t, 3H, CH₃), 1.98 (q, 2H, CH₂CH₃), 2.30 (s, 3H, CH₃), 2.25 (m, 2H, CH₂CH₂—CH), 2.40 (t,_{—}2H, CH₂CH₂—CH) 2.69 (m, 10H, CH₂), 3.54 (s, 2H, CH₂COOH), 3.85 (s, 1H, O—CH₃), 3.53 (m, 12H, All cis), 6.70 (d, 1H, Aromatic), 7.05 (s, 1H, Aromatic), 7.32 (d, 1H, Aromatic).

### Synthesis of N-(docosa-4,7,10,13,16,19-hexaenoyl)-3-amino-phenyl-acetic acid (MWL073)

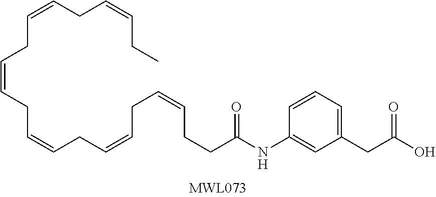 [SMILES: CC/C=C\C/C=C\C/C=C\C/C=C\C/C=C\C/C=C\CCC(=O)Nc1cccc(CC(=O)O)c1]

N-(docosa-4,7,10,13,16,19-hexaenoyl)-3 -amino-phenyl-acetic acid (MWL073) was synthesized according to the general procedures described hereinabove, wherein the fatty acid is DHA and the amine-containing compound is 3-amino-phenyl-acetic acid.

¹H-NMR(CDCl₃): 1.02 (t, 3H, CH₃), 2.05 (q, 2H, CH₂CH₃), 2.33-2.37 (dd, 4H, CH₂—CH₂), 2.65 (m, 10H, CH₂), 3.70 (s, 2H, CH₂—COOH), 5.35 (m, 12H, All-cis), 7.00-7.75 (m, 4H, Aromatic).

### Synthesis of N-docosa-4,7,10,13,16,19-hexaenoyl-piperidine-3-carboxylic acid (MWL074)

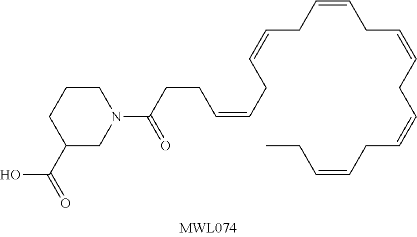 [SMILES: CC/C=C\C/C=C\C/C=C\C/C=C\C/C=C\C/C=C\CCC(=O)N1CCCC(C(=O)O)C1]

N-docosa-4,7,10,13,16,19-hexaenoyl-piperidine-3-carboxylic acid (MWL074) was synthesized according to the general procedures described hereinabove, wherein the fatty acid is DHA and the amine-containing compound is piperidine-3-carboxylic acid (nipecotic acid).

¹H-NMR(CDCl₃): 1.00 (t, 3H, CH₃), 1.18-2.21 (m, 6H, CH₂-Pipiridine Ring), 2.25 (q, 2H, CH₂CH₃), 2.33-2.37 (dd, 4H, CH₂—CH₂), 2.40 (m, 1H, CH-Pipiridine Ring), 2.65 (m, 10H, CH₂), 3.70-3.44 (m, 2H, CH₂-Pipiridine Ring), 5.35 (m, 12H, All-cis).

### Synthesis of N-docosa-4,7,10,13,16,19-hexaenoyl-1,2,3,6-tetrahydropyridine-4-carboxylic acid (MWL075)

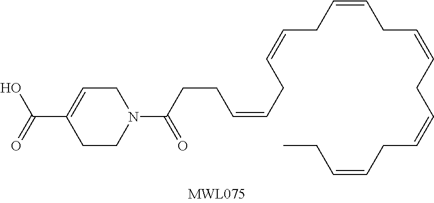 [SMILES: CC/C=C\C/C=C\C/C=C\C/C=C\C/C=C\C/C=C\CCC(=O)N1CC=C(C(=O)O)CC1]

N-docosa-4,7,10,13,16,19-hexaenoyl-1,2,3,6-tetrahydro-pyridine-4-carboxylic acid

(MWL075) was synthesized according to the general procedures described hereinabove, wherein the fatty acid is DHA and the amine-containing compound is 1,2,3,6-tetrahydropyridine-4-carboxylic acid (isoguvacine).

¹H-NMR(CDCl₃): 1.09 (t, 3H, CH₃), 2.02 (q, 2H, CH₂—CH₃), 2.11 (m, 2H, CH₂— Pipiridine Ring), 1.18-2.21 (m, 6H, CH₂-Pipiridine Ring), 2.33-2.37 (dd, 4H, CH₂-CH₂), 2.65 (m, 10H, CH₂), 3,56 (t, CH₂, Pipiridine Ring), 3.95 (d, 2H, CH₂— Pipiridine Ring), 5.38 (m, 12H, All-cis), 7.34 (d, 1H, CH— Pipiridine Ring).

### Synthesis of 5-(N-docosa-4,7,10,13,16,19-hexaenoyl-aminomethyl)-4,5-dihydroisoxazol-3-ol (MWL076)

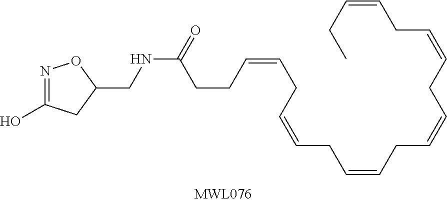 [SMILES: CC/C=C\C/C=C\C/C=C\C/C=C\C/C=C\C/C=C\CCC(=O)NCC1CC(O)=NO1]

5-(N-docosa-4,7,10,13,16,19-hexaenoyl-aminomethyl)-4,5-dihydroisoxazol-3-ol (MWL076) is synthesized according to the general procedures described hereinabove, wherein the fatty acid is DHA and the amine-containing compound is 5-(aminomethyl)-4,5-dihydroisoxazol-3-ol (4,5-dihydromuscimol).

### Synthesis of 5-(docosa-4,7,10,13,16,19-hexaenoyl)-3a,4,5,6,7,7a-hexahydro-isoxazolo[4,5-c]pyridin-3-ol (MWL077)

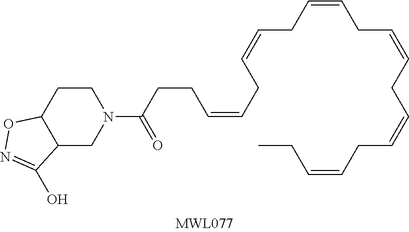 [SMILES: CC/C=C\C/C=C\C/C=C\C/C=C\C/C=C\C/C=C\CCC(=O)N1CCC2ON=C(O)C2C1]

5-(docosa-4,7,10,13,16,19-hexaenoyl)-3a,4,5,6,7,7a-hexahydro-isoxazolo [4,5-c]pyridin-3-ol (MWL077) is synthesized according to the general procedures described hereinabove, wherein the fatty acid is DHA and the amine-containing compound is 3a,4,5,6,7,7a-hexahydro-isoxazolo [4,5-c]pyridin-3-ol.

### Synthesis of 5-(docosa-4,7,10,13,16,19-hexaenoyl)-4,5,6,7-tetrahydro-isoxazolo[4,5-c]pyridin-3-ol (MWL078)

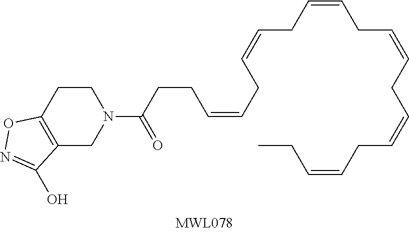 [SMILES: CC/C=C\C/C=C\C/C=C\C/C=C\C/C=C\C/C=C\CCC(=O)N1CCc2onc(O)c2C1]

5-(docosa-4,7,10,13,16,19-hexaenoyl)-4,5,6,7-tetrahydro-isoxazolo[4,5 -c]pyridin-3 -ol (MWL078) is synthesized according to the general procedures described hereinabove, wherein the fatty acid is DHA and the amine-containing compound is 4,5,6,7-tetrahydro-isoxazolo[4,5-c]pyridin-3-ol.

### Synthesis of 4-(3,5-dihydroxystylyl)phenyl docosa-4,7,10,13,16,19-hexaenoate (MWL080)

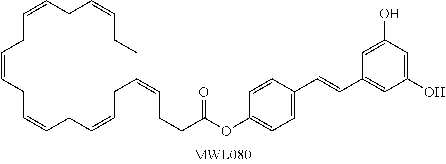 [SMILES: CC/C=C\C/C=C\C/C=C\C/C=C\C/C=C\C/C=C\CCC(=O)Oc1ccc(/C=C/c2cc(O)cc(O)c2)cc1]

4-(3,5-dihydroxystyryl)phenyl docosa-4,7,10,13,16,19-hexaenoate (MWL080) is synthesized according to the general procedures described hereinabove, wherein the fatty acid is DHA and the alcohol-containing compound is 3,5,4′-trihydroxystilbene (resveratrol).

### Synthesis of O-(γ-linolenoyl)-salicylic acid (MWL014)

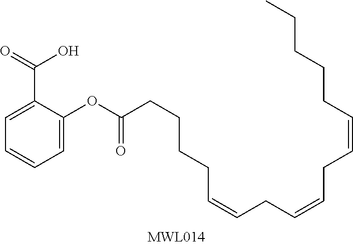 [SMILES: CCCCC/C=C\C/C=C\C/C=C\CCCCC(=O)Oc1ccccc1C(=O)O]

O-(γ-linolenoyl)-salicylic acid (MWL014) is synthesized according to the general procedures described hereinabove, wherein the fatty acid is γ-linolenic acid (GLA) and the alcohol-containing compound is salicylic acid.

### Synthesis of N-(γ-linolenoyl)-5-amino-salicylic acid (MWL015)

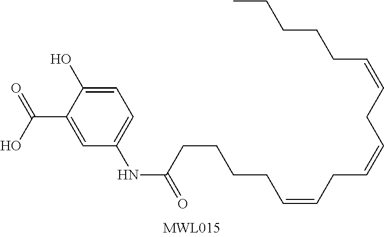 [SMILES: CCCCC/C=C\C/C=C\C/C=C\CCCCC(=O)Nc1ccc(O)c(C(=O)O)c1]

N-(γ-linolenoyl)-5-amino-salicylic acid (MWL015) is synthesized according to the general procedures described hereinabove, wherein the fatty acid is γ-linolenic acid (GLA) and the amine-containing compound is 5-amino-salicylic acid (mesalazine).

### Synthesis of N-oleoyl-5-hydroxy-indol-3-yl-acetic acid (MWL007)

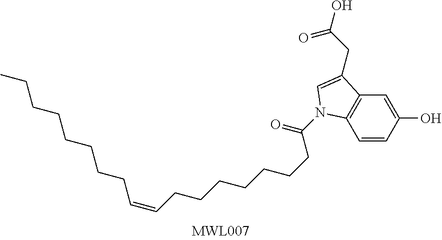 [SMILES: CCCCCCCC/C=C\CCCCCCCC(=O)n1cc(CC(=O)O)c2cc(O)ccc21]

N-oleoyl-5-hydroxy-indol-3-yl-acetic acid (MWL007) was synthesized according to the general procedures described hereinabove, wherein the fatty acid was oleic acid and the amine-containing compound was 5-hydroxy-indol-3-yl-acetic acid.

¹H-NMR(CDCl₃): 0.89 (t, 3H, CH₃), 1.29-1.31 (s, 2H, CH₂), 1.60 (t, 2H, N—CON—CH₂13 CH₂), 2.201 (m, 4H, CH—CH₂), 2.43 (t, 2H, NCOCH₂), 3.58 (s, 2H, CH₂COOH), 5.49 (dd, 2H, CH—CH-cis), 7.33 (s, 1H, NCHCH₂-Ring), 7.42-7.55 (m, 3H, Aromatic).

### Synthesis N-oleoyl-2-amino-nicotinic acid (MWL008)

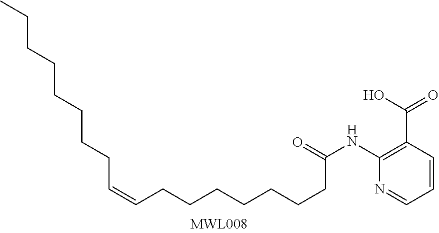 [SMILES: CCCCCCCC/C=C\CCCCCCCC(=O)Nc1ncccc1C(=O)O]

N-oleoyl-2-amino-nicotinic acid (MWL008) was synthesized according to the general procedures described hereinabove, wherein the fatty acid was oleic acid and the amine-containing compound was 2-amino-nicotininc acid.

¹H-NMR (CDCl₃): 0.93 (t, 3H, CH₃), 1.29-1.35 (s, 20H, CH₂), 1.62 (t, 2H, N—CON—CH₂—CH₂), 2.20 (m, 4H, CH—CH₂), 2.45 (t, 2H, NCOCH₂), 3.53 (s, 2H, CH₂COOH), 5.45 (dd, 2H, CH—CH-cis), 7.88-8.55 (m, 3H, Aromatic).

### Synthesis N-oleoyl-salicylic acid (MWL009)

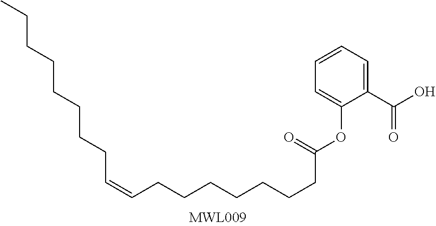 [SMILES: CCCCCCCC/C=C\CCCCCCCC(=O)Oc1ccccc1C(=O)O]

N-oleoyl-salicyclic acid (MWL009) was synthesized according to the general procedures described hereinabove, wherein the fatty acid was all-cis-DHA and the alcohol-containing compound was salicylic acid.

¹H-NMR (CDCl₃): 0.85 (t, 3H, CH₃), 1.29-1.39 (s, 20H, CH₂), 1.62 (t, 2H,—CH₂—CH₂), 2.22 (m, 4H, CH—CH₂), 2.46 (t, 2H, NCOCH₂), 3.50 (s, 2H, CH₂COOH), 5.45 (dd, 2H, CH—CH-cis), 7.91-8.20 (m, 4H, Aromatic).

### Synthesis of N-oleoyl-5-amino-salicylic acid (MWL017)

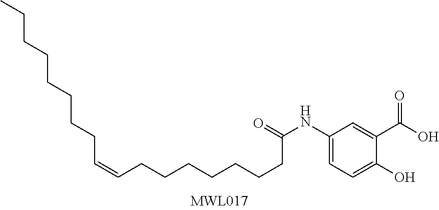 [SMILES: CCCCCCCC/C=C\CCCCCCCC(=O)Nc1ccc(O)c(C(=O)O)c1]

N-oleoyl-5-amino-salicylic acid (MWL017) was synthesized according to the general procedures described hereinabove, wherein the fatty acid is oleic acid and the amine-containing compound is 5-amino-salicylic acid (mesalazine).

¹H-NMR (CDCl₃): 0.88 (t, 3H, CH₃), 1.23-1.33 (s, 20H, CH₂), 1.59 (t, 2H,—CH₂—CH₂), 2.20 (m, 4H, CH—CH₂), 2.49 (t, 2H, NCOCH₂), 3.47 (s, 2H, CH₂COOH), 5.49 (dd, 2H, CH—CH-cis), 7.41-8.28 (m, 3H, Aromatic).

### Synthesis of N-docosa-4,7,10,13,16,19-hexaenoyl-taurine (MWL002)

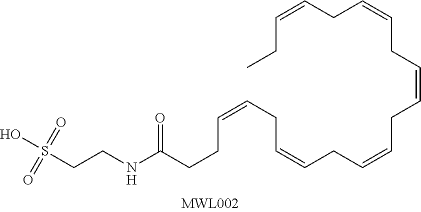 [SMILES: CC/C=C\C/C=C\C/C=C\C/C=C\C/C=C\C/C=C\CCC(=O)NCCS(=O)(=O)O]

0.54 ml of triethylamine was added to a solution of 2 grams of all-cis-docosa-4,7,10,13,16,19-hexaenoic acid (all-cis-DHA) in 20 ml of dry tetrahydrofuran (THF), and the mixture was stirred for 3 minutes at room temperature. Triethylchloroformate was then added, resulting in the formation of a white participate. A solution of 1 gram of taurine in 2 ml water was added, and the mixture was stirred overnight at room temperature. A pale yellow solution was obtained. The THF was removed via evaporation. The product was purified by column chromatography using a mixture of 1:2 hexane:ethyl acetate. A purity of 98% N-docosa-4,7,10,13,16,19-hexaenoyl-taurine was obtained, as determined by high performance liquid chromatography (HPLC) and thin-layer chromatography (TLC).

The structure of the product was confirmed by ¹H-NMR spectroscopy and by liquid chromatography-mass spectroscopy (LC-MS).

¹H-NMR (CDCl₃): 1.05 (t, 3H, CH₃), 2.01 (q, 2H, CH₂CH₃), 2.23-2.26 (d, 4H, CH₂CH₂), 2.63 (m, 10H, CH₂), 3.66 (t, 2H, CH₂—SO₃H), 3.78 (t, 2H, CH₂NH), 5.45 (m, 12H, All-cis).

### Synthesis of MWL001

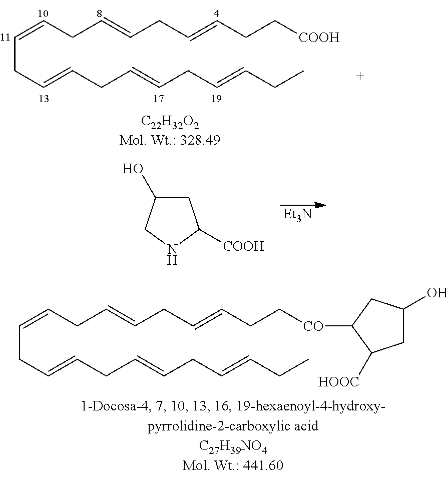 [SMILES: "Et3N" ; CC/C=C/C/C=C/C/C=C/C/C=C\C/C=C/C/C=C/CCC(=O)O ; O=C(O)C1CC(O)CN1 ; CC/C=C/C/C=C/C/C=C/C/C=C\C/C=C/C/C=C/CCC(=O)C1CC(O)CC1C(=O)O]

Solution (i): A solution of 300 milliliter dry THF and 10 gr. (0.03 mol) Of Decosahexanoic acid (DHA) was prepared (at 4 0C). To this solution; four milliliter of Triethylcloroformate were added and the mixture was stirred for 30 min (at 4 0C). Then a 5 ml of Triethylamine (dissolved in 50 of dry THF) were slowly added resulting in formation of a white participate (with vigorous stirring). The reaction was stirred for 3 hour at room temperature and filtered through filter paper to a clean flask.

Solution (ii): NaOH (1 gram) was dissolved in 20 ml of water (DDW). To this solution; 5 grams of Hydroxyproline were added and stirred vigorously to get a clear solution. After filtration; solution (i) was stirred vigorously and solution (ii) was added. The mixture was stirred overnight at RT.

After 24 hours; a 200 milliliter 10% HCl solution was added to the mixture; stirred for 30 minutes. Then a 300 milliliter of hexane was added. The aqueous (lower) layer was discarded. The upper layer was collected, washed twice with 200 of brine. The organic layer was collected; dried over anhydrous sulfate (10 grams); filtered through a filter paper. The organic solution was evaporated till dryness. A 70% yield was achieved after column chromatography.

### Example 3

### Safety Studies

hERG is a cardiac channel, commonly used in models for testing cardiological adverse side effects of potential therapeutically active agents.

The sensitivity of hERG channels to two compounds, MWL001 also termed in the application as MW001 or MWL-001 and MWL002 also termed MW002 or MWL-002, was tested using the Xenopus oocyte expression system and the two-electrode voltage clamp technique. Activity of the compounds was tested at a concentration of 15 μM both from the external and from the internal sides of the membrane.

Materials and Methods:

Chemicals: MWL001 (MW=441.6) was dissolved in ethanol to prepare a stock solution of 6.62 mg/ml (15 mM). MWL002 (MW=435.6) was dissolved in DMSO to prepare a stock solution of 1.1 mg/ml (2.5 mM).

Clones: hERG gene (gi|26051269) was cloned within pSP64 expression vector (Promega) downstream to a SP6 RNA polymerase promoter. cRNA was prepared following digestion with EcoR I.

Electrophysiology: Xenopus laevis oocytes were isolated, defolliculated and maintained at 17° C. in ND-91 solution (in mM): 91 NaCl, 2 KCl, 1.8 CaCl₂, 1 MgCl₂, and 5 HEPES, pH 7.5, supplemented with 100 U/ml penicillin, 100 μg/ml streptomycin and 50 μg/ml gentamycin. Oocytes were injected with 20 or 40 nl, containing 7-14 ng of hERG cRNA. Whole-cell currents were measured 2-3 days after injection by the two-electrode voltage clamp technique (TEVC), using a GeneClamp 500B amplifier (Axon Instruments). Data were filtered at 2 kHz and sampled at 5 kHz with Clampex 9.0 software (Axon Instruments). The pipette was filled with 3M KCl and the bath solution contained (in mM): 4 KCl, 96 NaCl, 1 MgCl₂, 0.3 CaCl₂, 5 HEPES, pH 7.4. For external application of the compounds, stock solutions of either MWL001 or MWL002 were dissolved in the bath solution to a final concentration of 15 μM. During the experiments, bath solution exchange rate was about 2 ml/minutes. At this rate, more than 95% of the bath solution is replaced within 30 seconds. For internal blockade measurements, oocytes were injected with 46 nl of diluted (0.274 mM) compound solutions to achieve a final concentration within the oocyte of 15 μM.

Results:

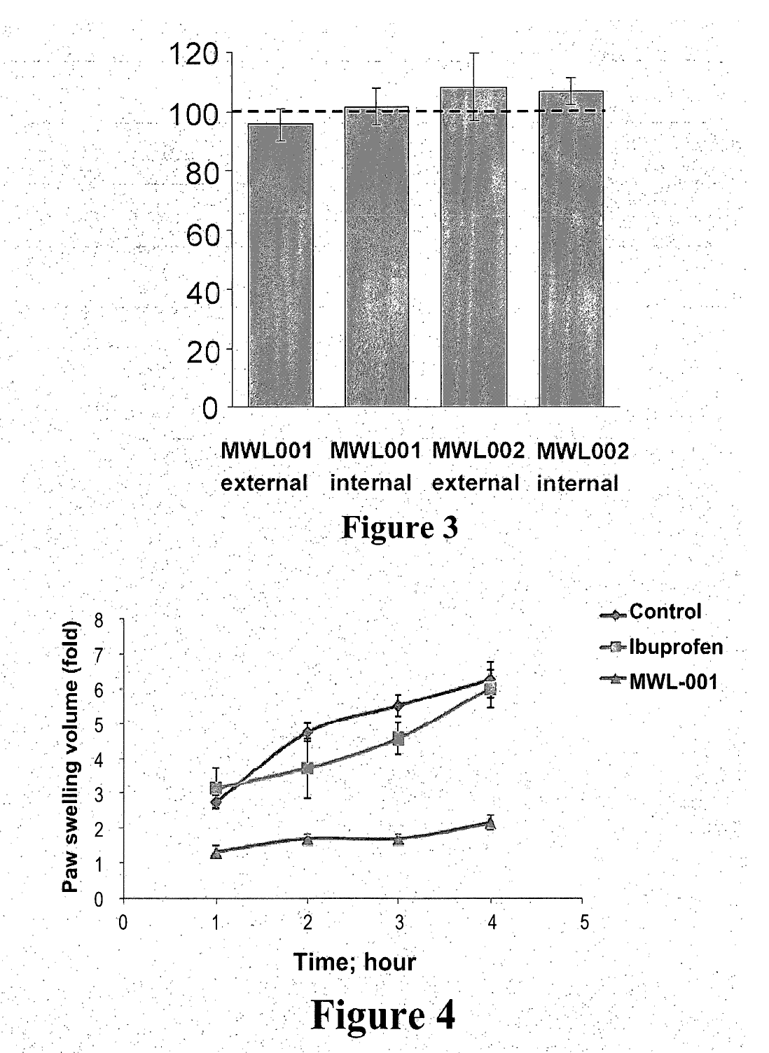

Internal application: Compounds were injected into the oocyte to achieve a final concentration of 15 μM. Injection was made during current measurements and results were collected before and for 5 minutes after the injection. Current levels were determined in 4 seconds intervals at −130 mV, following a pre-pulse to +40 mV (see, Table 4 and FIG. 3).

In addition, measurements were made to determine channel behavior throughout the entire physiological voltage range. An example of such analysis is presented in FIGS. 2A to 2C.

FIGS. 2A-D further present results from a representative oocyte expressing hERG channels before and after injection with MWL002 (15 μM). FIG. 2A presents currents before injection. FIG. 2B presents currents 5 minutes after injection. From a holding potential of −80 mV, the oocyte was pulsed from −100 to +70 mV in 15 mV voltage steps for 80 ms, with 2 seconds interpulse intervals and then pulsed to −130 mV for 80 ms. FIG. 2C presents voltage activation curves for the oocyte measured in FIGS. 2A and 2B. Presented are normalized currents at −130 mV that were initiated by the indicated voltage. FIG. 2D presents currents during 6-minute-long measurements prior and post injection of MLW002, as indicated by the gray bar. Currents were initiated by a 150-ms-long pulse to +40 and measured at −130 mV, with 15 seconds interpulse intervals.

External application: MWL compounds were applied at 15 μM concentration in the bath solution and current levels were determined in 4 seconds intervals at −130 mV, following a pre-pulse to +40 mV, for 15 minutes (see, Table 4). In addition, measurements were made to determine channel behavior throughout the entire physiological voltage range.

Table 4 presents the data obtained for the effect of MWL compounds on expressed hERG channels in individual oocytes which were either incubated (external) or injected (internal) with MWL compounds. Currents were monitored for 15 or 5 minutes, respectively.

TABLE 4

| | experiment | initial | final | remaining | | |
|---|---|---|---|---|---|---|
| sample | # | current | current | current | avarage | SD |
| MWL001- | 1 | −12.7 | −12.8 | 100.8 | 95.7 | 5.5 |
| external | 2 | −17.0 | −15.5 | 91.2 | | |
| | 3 | −16.1 | −15.8 | 98.1 | | |
| | 4 | −10.8 | −10.8 | 100.0 | | |
| | 5 | −29.3 | −26.0 | 88.6 | | |
| MWL001- | 1 | −6.8 | −6.5 | 95.6 | 101.6 | 6.4 |
| internal | 2 | −10.9 | −11.1 | 101.8 | | |
| | 3 | −13.8 | −15.5 | 112.3 | | |
| | 4 | −9.5 | −9.5 | 100.0 | | |
| | 5 | −18.8 | −18.5 | 98.4 | | |
| MWL002- | 1 | −7.8 | −7.1 | 91.6 | 108.2 | 11.4 |
| external | 2 | −4.6 | −5.0 | 109.9 | | |
| | 3 | −6.0 | −7.2 | 120.0 | | |
| | 4 | −17.5 | −18.0 | 102.9 | | |
| | 5 | −7.7 | −9.0 | 116.9 | | |
| MWL002- | 1 | −12.5 | −13.7 | 109.6 | 106.9 | 4.5 |
| internal | 2 | −6.3 | −6.5 | 103.2 | | |
| | 3 | −11.3 | −11.5 | 101.8 | | |
| | 4 | −6.7 | −7.2 | 107.5 | | |
| | 5 | −7.1 | −8.0 | 112.7 | | |

FIG. 3 presents average changes in expressed hERG currents. Values were taken from Table 4.

In all measurements, no effect of MWL compounds on hERG channels was observed.

Accordingly, it has been demonstrated that the exemplary tested conjugates do not exhibit any detectable effect on hERG channel currents.

### Example 4

### COX-1 and COX-2 Inhibition Measurements

Exemplary conjugates as described herein were tested in vitro for inhibition of COX-1 and COX-2, using an enzyme immunoassay (EIA), as follows.

Cyclooxygenase catalyzes the first step in the biosynthesis of arachidonic acid (AA) to PGH2. PGF2α, produced from PGH2 by reduction with stannous chloride, is measured by enzyme immunoassay (ACETM competitive EIA).

Stock solutions of test compounds were dissolved in a minimum volume of DMSO. Briefly, to a series of supplied reaction buffer solutions (960 μl, 0.1M Tris-HCl pH 8.0 containing 5 mM EDTA and 2 mM phenol) with either COX-1 or COX-2 (10 μl) enzyme in the presence of heme (10 μl) were added 10 μl of various concentrations of test drug solutions (0.01, 0.1, 1, 10, 50, and 100 μM in a final volume of 1 ml). These solutions were incubated for a period of 5 minutes at 37° C. after which 10 μL of AA (100 μM) solution were added and the COX reaction was stopped by the addition of 50 μl of 1M HCl after 2 minutes. PGF2α, produced from PGH2 by reduction with stannous chloride was measured by enzyme immunoassay.

This assay is based on the competition between PGs and a PG-acetylcholinesterase conjugate (PG tracer) for a limited amount of PG antiserum. The amount of PG tracer that is able to bind to the PG antiserum is inversely proportional to the concentration of PGs in the wells since the concentration of PG tracer is held constant while the concentration of PGs varies. The concentration of the test compound causing 50% inhibition (IC50, μM) was calculated from the concentration-inhibition response curve (duplicate determinations).

Table 5 below presents the results obtained for exemplary conjugates.

TABLE 5

| | IC50 (mM) | | |
|---|---|---|---|
| Compound | COX-1 | COX-2 | Ratio (COX ½) |
| MWL-001 | 39.45 | 0.085 | 464.11 |
| MWL-002 | 0.780 | 0.028 | 27.86 |
| MWL-020 | 0.102 | 0.017 | 6 |
| MWL-007 | 0.380 | 0.024 | 15.83 |
| MWL-021 | 0.325 | 0.009 | 36.11 |

### Example 5

### Anti-Inflammation Studies

Exemplary conjugates were subjected to in vivo study of paw edema measurements in rats. Sprague dawley rats (150-200 grams) were used. Edema was induced by a single sub-plantar injection of carrageenan (1 mg/paw) into the left hind paw of the rat under light ether anesthesia. The total volume injected was 0.1 ml. The paw volume was measured immediately before the injection and at hourly intervals thereafter using a hydroplethysmometer (model 7150, Ugo Basile, Italy). The results were expressed either as the increase in paw volume (ml) calculated by subtracting the basal volume or by calculating the area under the time-course curve (AUC; ml h) for each group.

The anti-inflammatory activity of the conjugates was tested versus Ibuprofen, as a reference, on carrageenan-induced edema at different time intervals.

The results obtained for an exemplary conjugate, MLW001 are presented In FIG. 4, and clearly show an anti-inflammatory effect of the tested conjugates, superior to Ibuprofen.

### Example 6

### TNBS-Induced Colitis

Exemplary conjugates were tested for the effect on inflammatory bowel disease IBD (ulcerative colitis) in rats.

Briefly, ulcerative colitis was induced by tri-nitrobenzene sulfunic acid (TNBS) as described in the literature. Rats were divided into 4 groups, (−) sham (healthy), (+) control ulcerative colitis (UC, not treated), UC treated with the tested conjugate (25 mg/kg), and UC treated with 5-amino salicylic acid (5-ASA 25 mg/kg). The tested conjugate and 5-ASA were administered rectally during all the period of the experiment. At the end of the experiment animals were sacrificed and the colon was isolated to test the severity of the inflammation. The severity of the inflammation was tested by measurement of the myeloperoxidase activity (MPO activity) in the inflamed area of the colon.

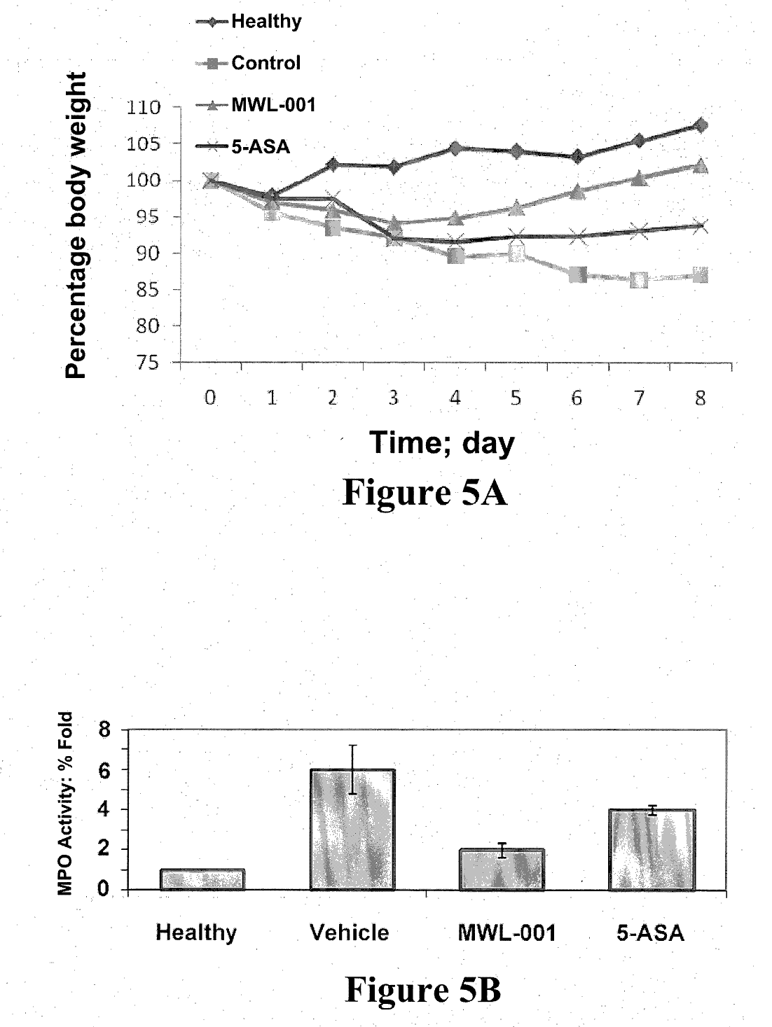

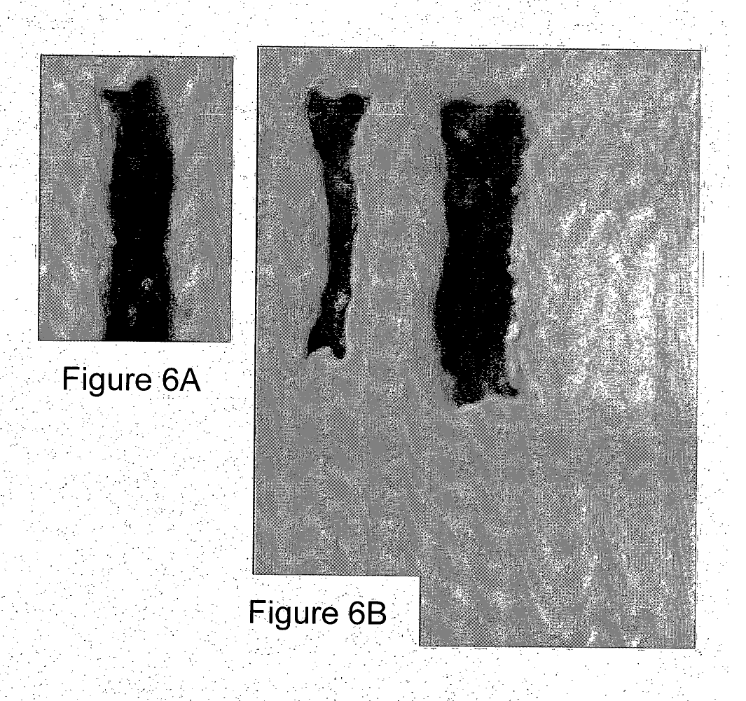

The results obtained for MWL-001, as an exemplary tested conjugate are presented in FIGS. 5A and 6B. FIG. 5A presents the change in body weight throughout the assay period. FIG. 5B presents the data obtained for the severity index. MWL-001 (50 mg/kg) showed a significant decrease in MPO activity when compared to NSAID; 5-Aminosalicylic acid (5-ASA); with no decrease in the body weight.

FIGS. 6A and 6B present images of untreated (FIG. 6A), and MWL-001-treated and 5-ASA-treated (FIG. 6B) ulcerated colon segments. Colon treated with MWL-001 has a normal clear anatomic morphology and a significant decrease in inflammation signs compared to those non-treated or treated with 5-ASA.

### Example 7

### Collagen Induced Arthritis (CIA)

Male black/57 mice, age 8-10 weeks, were used in in vivo study of PGE2 production and TNFα levels in collagen induced arthritis (CIA) model.

Bovine CII (type II collagen CII, Sigma, St. Louis, Mo., USA) was dissolved in 0.1 M acetic acid overnight at 4° C. and was thereafter emulsified in an equal volume of complete Freund's adjuvant (Sigma). The mice were immunized intradermally at the base of the tail with 100 μl of emulsion containing 100 μg of CII. On day 21, mice were boosted intraperitoneally with 100 μg CII dissolved in phosphate buffered saline (PBS).

The tested conjugate or Ibuprofen (Sigma, Israel) were dissolved in 80% Cremophor EL:saline 80%:20%, respectively. Treatment was commenced from the first day of the onset of the clinical symptoms of arthritis, which was considered to be the day when the first visible signs of erythema and/or oedema were observed in any of the limbs.

Mice were randomly selected and assigned to one of the following groups: tested conjugate (250 mg/(kg/day); n=4), Iboprofen (500 mg/ (kg/day); n=4) or vehicle (n=4). The tested conjugate and Ibuprofen were administered orally. Treatment was given daily for a period of 21 days.

Joint tissues were prepared as previously described for measuring the production of PGE2 and cytokines. Briefly, the left forepaw (including the paw, ankle, and knee) from each mouse was removed and homogenized in 100 mg tissue/1 ml of lysis medium (75% ethanol in 0.1 M sodium acetate, adjusted to pH 3 with HCl for PGE2, and RPMI 1640 containing 2 mM phenylmethylsulfonyl fluoride and 1 mg/ml of aprotinin, leupeptin, and pepstatin A for cytokines). The homogenates were then centrifuged 3500×g for 15 minutes at 4° C. Sera were obtained from the mice on day 22 of arthritis, as described above. Supernatants and sera were stored at −20° C. until use. PGE2 concentration was measured with a commercial radio immunoassay (RIA) kit (Amersham, UK) according to the manufacturer's instructions. Commercial enzyme-linked immunosorbent assay kit was used to measure the concentrations of TNFα (Diaclone, France) in serum according to the manufacturer's instructions. Results were expressed as pg/ml of serum or supernatant from joint homogenate.

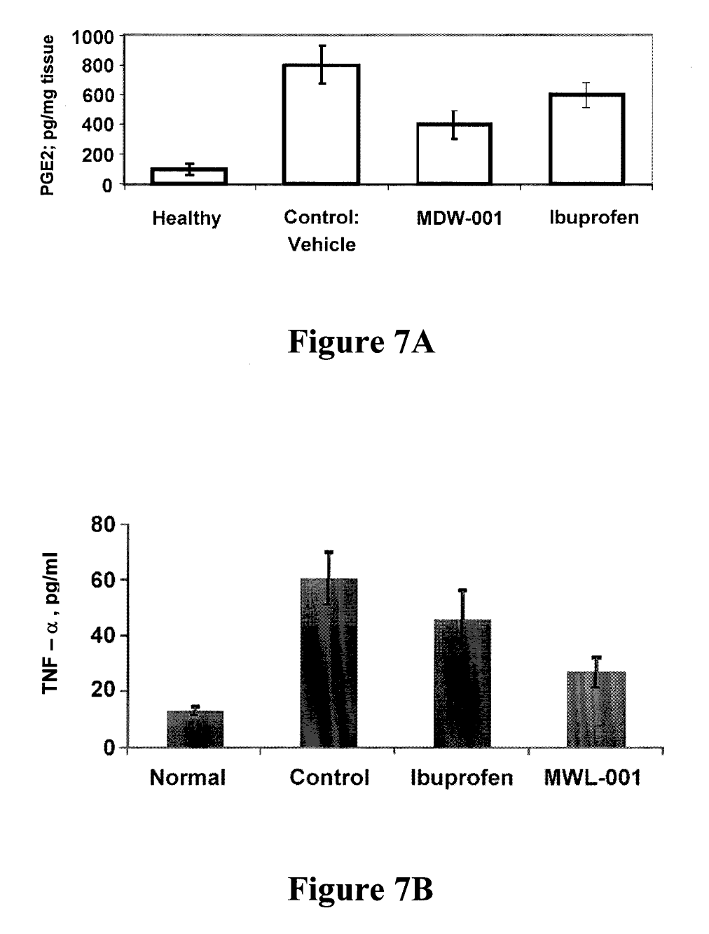

The data obtained for MWL-001 is presented in FIGS. 7A and 7B. Treatment with MWL-001 shows a significant decrease in Prostaglandin E2 and TNF-α levels in the joints compared to Ibuprofen treatment.

In a different study, 34 male 8-10 weeks old DBA/1J mice were used. Animals were fed ad libitum a commercial rodent diet (Teklad Certified Global 18% Protein Diet cat #: 106S8216). Animals had free access to acidified drinking water (pH between 2.5 and 3.0) obtained from the municipality supply.

The effect of MWL-002 (conjugate of DHA and taurine) was tested and compared to that of Ibuprofen. Cremophore EL was used as a vehicle.

Mice were divided to the following groups:

| Group | | | |
|---|---|---|---|
| (Starting No. of Animals) | | | |
| Animals numbers | Treatments (Daily) | volume | Route of Administration |
| 1M (n = 9) | 50 mg/kg DHA-Tau | 0.1 ml/10 g | Intra peritoneal injection |
| 1, 2, 3, 4, 33, 34, 35, 36, 38 | + Cremophore El | | |
| 2M (n = 9) | 50 mg/kg DHA | | |
| 5, 6, 7, 8, 29, 30, 31, 32, 39 | + Cremophore El | 0.1 m1/10 g | Intra peritoneal injection |
| 3M (n = 8) | Cremophore El | 0.1 m1/10 g | Intra peritoneal injection |
| 9, 10, 11, 12, 17, 18, 19, 20. | | | |
| 4M (n = 8) | 50 mg/kg Ibuprofen | 0.1 m1/10 g | Intra peritoneal injection |
| 13, 14, 15, 16, 25, 26, 27, 28 | | | |

On day 0, each mouse was anesthetized by inhalation of 3% isofluorane and injected intradermally at the base of the tail with 0.1 ml of emulsion containing 100 μg collagen, using a 1-ml glass syringe with a 26-guade needle. Booster injection was performed at day 15 to the same injection site.

The tested materials were administered 3 times a week, via IP for a total of 12 administrations starting on day 1.

Observations for clinical score were performed twice weekly until study termination.

The severity of arthritis was scored based on the level of inflammation in each of the four paws and recorded as one of five grades according to the following:

| Score | Grade (per paw) |
|---|---|
| 0 | No symptoms |
| 1 | Erythema and mild swelling confined to the tarsals or ankle joint. |
| 2 | Erythema and mild swelling extending from the ankle to the tarsals |
| | or ankle joint. |
| 3 | Erythema and mild moderate swelling extended from the ankle to |
| | metatarsal joints. |
| 4 | Erythema and severe swelling encompass the ankle, foot and digits, |
| | or ankylosis of the limb. |

Hind paw thickness (edema) was measured by digital caliper once a week until Day 14 and thereafter twice weekly and prior to study termination. Two cross sectional areas were marked, one on the paw and the other at the ankle (tarsal joint). Two measurements were made on each section, perpendicular to each other. The results obtained were calculated as the average areas of both hind limbs per animal.

Body weight was measured prior to dosing, during the study twice a week and prior to study termination.

At study termination the animals were sacrificed by Carbon dioxide asphyxiation. Clinical Results:

From the data obtained, it was shown that the body weight of the mice increased during the experiment. The body weight of the treated mice with 1M (DHA+Tau) and 3M (Ibuprofen) was higher in the first face of the experiment—days 12-16, From day 37 the treated group with DHA-Tau increase the body weight in a major percent, in comparison to the other 3 groups, and this trend continues until the end of the study.

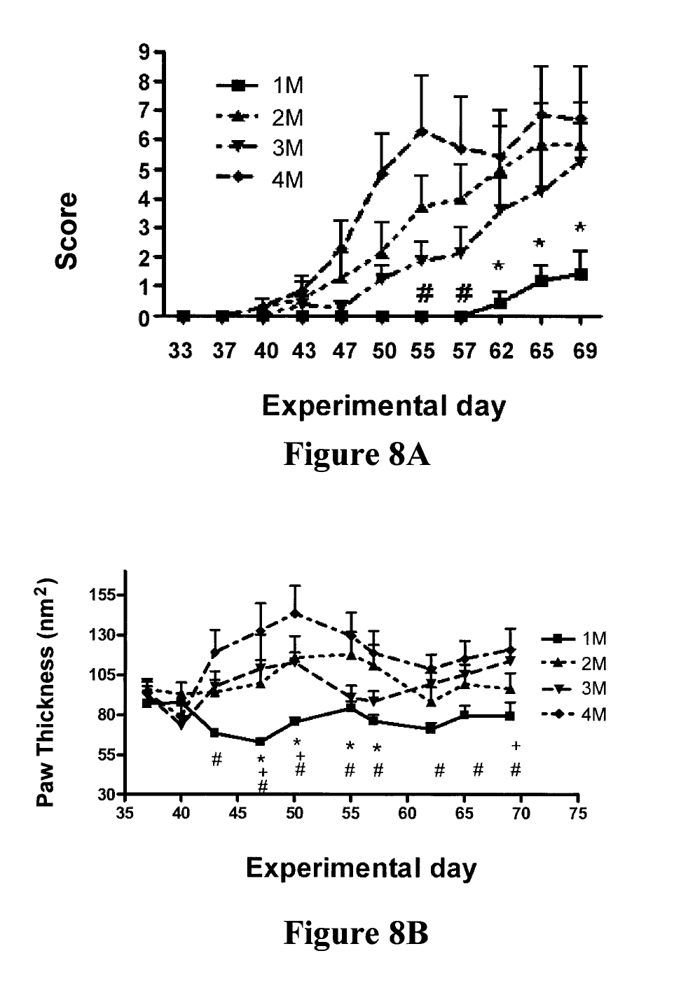

The arthtritis score (AS) of all the animals in the study was 0 until day 40. From day 40 until the end of the study the arthtritis score of the mice in groups 2, 3 and 4M increased daily. In group 1M (DHA-Tau) the AS initiate the increase in day 57 (at least 17 days after the other groups), as shown in FIG. 8A.

The arthtritis thickness index (ATI) of all the animals in the study was similar until day 40. The ATI of all the groups increased during the study (from day 0 to day 70) in 150% for group 1M, 180% for group 2M and 190% for groups 3 and 4 M (Paired t-test p<0.05,day 0 compared to day 70)

As demonstrated in FIG. 8B, from day 43 until the end of the study the ATI of the mice in groups 2, 3 and 4M increased daily at the time that almost no changes occurred in the treated group with DHA-Tau-1M.

The results of this study clearly show that DHA-Tau immunization inhibits CIA development robustly, where other compounds like DHA alone or Ibuprofen were not substantially effective.

The effect of DHA-Tau on the treated mice was verified by the different arthtritis parameters like arthtritis score, paw thickness, body weight and also by the latency of emergence of the first signs.

### Example 8

### Pharmacokinetics

Evaluation of the pharmacokinetic profile of MWL001 after oral administration in rats was performed.

Sprague-Dawley male rats weighing 300-325 g were used to evaluate the oral absorption of MWL001 and its blood levels. They were deprived of food overnight and given free access to water. A dose of 10 mg/kg MWL-001 (3.3 mg/rat) was dispersed in 2.5 mL DDW and delivered to the rats by oral gavage. Blood samples (300 μl) from the rats' tails were collected in heparin containing tubes at 0, 0.5, 1, 1.5, 2, 3, 4, 6, 8, 12 and 24 h. The samples were immediately centrifuged at 10,000 rpm for 5 min, after which 150 μl of plasma samples were transferred to new tubes and stored at −80° C. until analyzed by LCMS/MS.

The assay was based on protein precipitation of MWL001 and DHA (as an MWL001 metabolite) using methanol (100 μL plasma sample mixed with 900 ml of methanol. The separation was carried out under reverse-phase conditions employing a Phenomenex Synergi, column (MAX-RP, 50×2 mm, 2.5 μm, 100A (in gradient mode. The mobile phase A was methanol/water/formic acid 20/80/0.2 and the mobile phase B was methanol/water/formic acid 80/20/0.2. The flow rate was maintained at 0.3 mL/min, and the column was maintained at 40° C. HPLC-MS/MS analysis was performed with a Shimadzu LC-20 HPLC system coupled with a Sciex Qtrap 3200 Turbo Ion Spray detector in positive ionization mode. The tested samples were quantified against a calibration curve in the range of 5-50 ng/mL. The correlation coefficient values were better than 0.990 indicating that high linearity, accuracy and specificity were achieved.

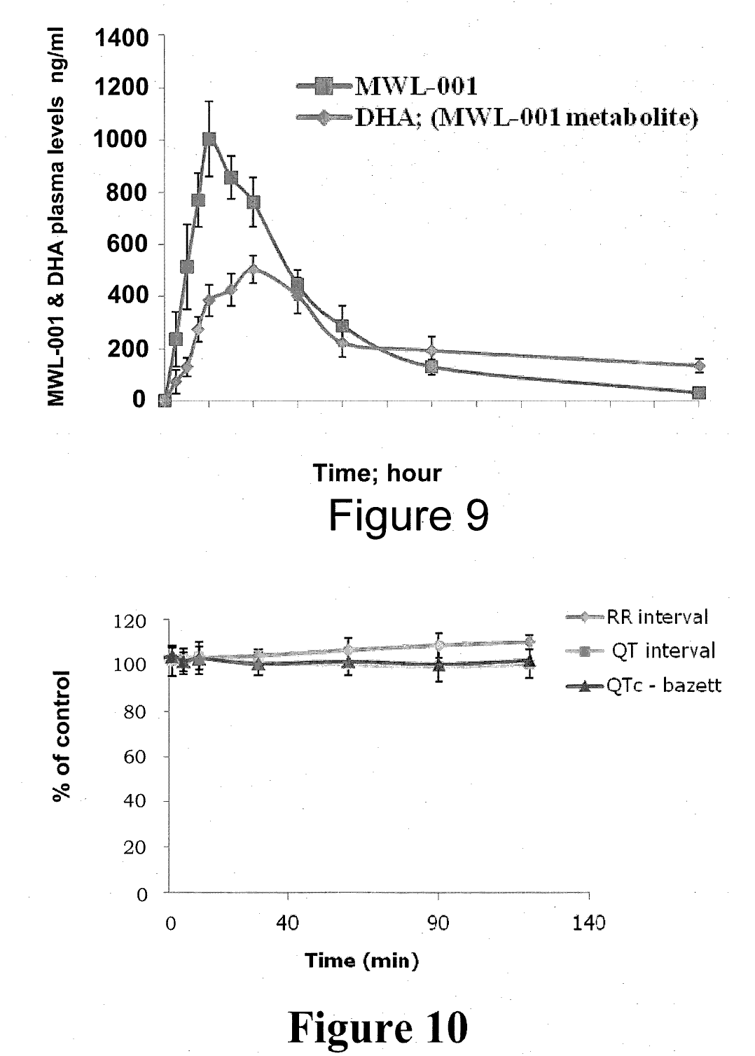

The pharmacokinetic profile of MWL001 after oral administration in rats is shown in FIG. 9. DHA, as MWL001 major metabolite; was also evaluated at the same time points, the results also depicted in FIG. 9.

The table below shows the pharmacokinetics parameters of MWL001:

| | |
|---|---|
| AUC (hr * ng/ml) | 6688.8 ± 837.7 |
| Tmax (hr) | 2.3 ± 0.5 |
| Cmax (ng/ml) | 1042.2 ± 77.5 |
| T½ (hr) | 4.9 ± 0.6 |
| Cl (Clearence) ml/hr/kg | 1512.7 ± 189.1 |
| V (Volume of distribution) | 11283.0 ± 3583.1 |

As shown in FIG. 9, a high levels of MWL001 was detected in the plasma indicating a high absorption from the gastrointestinal tract; hence a high bioavailability. Such results teach that MWL001 may be administered successfully orally.

Furthermore; the major metabolite of MWL001 was also evaluated at the same time points in the plasma; respectively to MWL001 itself (see FIG. 9). In FIG. 9 it is clearly shown that MWL001 undergoes decomposition to DHA, which is proven to be the major metabolite of the MWL001. MWL001 undergoes decomposition to DHA by the metabolic enzymes array of the intestine and the liver.

### Example 9

### Safety Data

### a. Acute Intraperitoneal (ip) Toxicity in the Mouse

The objective of this study was to determine the MTD (Maximum Tolerated Dose) and/or assess the potential toxic effects in terms of the MFD (Maximum Feasible Dose) following an acute intraperitoneal (IP) injection of the Test Item MWL002 (Batch No.: 201109) to male and female ICR mice, in consideration of its intended use as an anti-inflammatory and pain relief agent.

MWL002 (Batch No.: 201109) was injected at two dose levels of 1000 (corresponding to the MFD) and 200 mg/kg to two groups consisting of three male and three female ICR mice per group, by a single intraperitoneal (IP) injection. An additional equally-sized group was injected with vehicle and served as the Control group. Dosing was sequential using three male and three female mice per step at 14 days interval. The Test Item or Vehicle Control Dosing Solutions were freshly prepared by the Testing Facility on each day of dosing, by diluting the Test Item or Vehicle Control with Physiological Saline according to the appropriate dose level and a constant volume dosage of 4 ml/kg.

At the end of the 14-day observation period, the Vehicle Control group, the 200 mg/kg treated group and both survivors (females) of the 1000 mg/kg treated group exhibited mean body weight gain vs. the day of dosing. However, body weight loss was evident in all males and females of the 1000 mg/kg treated group and all males and one female of the 200 mg/kg treated group two days post dosing. Both survivors (females) of the 1000 mg/kg treated group and all males of the 200 mg/kg treated group regained their relative weight loss in the successive week.

In the 200 mg/kg treated group no gross pathological findings were evident in any of the animals at the time of their scheduled necropsy on study day 14. In the 1000 mg/kg treated group, gross lesions noted at the time of necropsy two and three days post dosing included hemorrhage-like lesions in stomach and intestinal walls (males 114 &. 6 and female //14) and enlarged & red-colored mesenteric lymph node (male #5 and female #14). No gross pathological findings were evident in any of the surviving two females at the time of their scheduled necropsy on study day 14. In the Vehicle Control treated animals, gross pathological findings at the time of their scheduled necropsy on study day 14 were confined to uneven color of liver found in one male.

Under the conditions of this study and in view of the results obtained following a single intraperitoneal (IP) injection of the Test Item MWL002 (Batch No.: 201109), to male and female ICR mice at two dose levels of 1000 mg/kg and 200 mg/kg, it may be concluded that the MTD of the Test Item is higher than 200 mg/kg and lower than 1000 mg/kg.

### b. Cardio Toxicity: QT Prolongation- MWL001

The QT interval (time from beginning of the QRS complex and to the end of the T wave) of the ECG is a measure of the duration of ventricular depolarization and repolarization. When ventricular repolarization is delayed and the QT interval is prolonged, there is an increased risk of ventricular tachyarrhythmia, including torsade de points (potentially fatal polymorphic ventricular tachycardia). As the QT interval has an inverse relationship to heart rate, the measured QT intervals are generally corrected for heart rate in order to determine whether they are prolonged relative to the baseline. Various correction formulae have been suggested, of which Bazzet's and Fridericia's correction are the most widely used.

The initial evaluation of cardiac safety by ECG measurements focuses on QTc duration of MWL001 50 mg/ml concentration.

Quinidine (QND) hydrochloride and urethane was purchased from Sigma (Israel).

Male Sprague-Dawley rats weighting 250-300 g were purchased from Harlan (Israel) and used for the experiment.

Male Sprague-Dawley rats (280-300 g of weight) were used for electrocardiographic measurements at basal conditions as well as medicated with QND and MWL. The rats were anesthetized with urethane (0.5 gr/kg, intraperitoneally). Electrocardiograms were recorded using disk electrodes fitted subcutaneously near the right and left axial regions and at the xiphoid cartilage and secured by surgical clips for standard Lead II recording. The left femoral vain and artery were cannulated with a polyethylene tubes (PE-50, Intramedic) for blood pressure recording and drug administration. The artery cannula was connected to a blood pressure transducer (Biometrix, Israel), the vain cannula were connected to a syringe infusion pump (Harvard apparatus 22, USA). All of the data was recorded (AdInstruments PowerLab 16/30, Australia) for off line analysis.

Heart rate (RR interval), QT interval and QTc (corrected QT interval) were calculated by ECG analysis module in the LabChart 7.2.1 software and manually screened by trained technicians. The data points selected as control were 10 min before injection, 1, 5, 10, 30, 60, 90, 120, 150, 180 min after injection. At each data point, ˜1 min of ECG data was manually selected; the ECG parameters were measured by aligning 6-4 consecutive beats by the software till the end of the selection, the software detection was manually validated and averaged. The corrected QT interval (QTc) was calculated by the Bazett formula (QT/RR^{0.5}) by the software (according the FDA-TDP concept paper E14, S7B 2008), the correction was calculated by the Fridericia formula (QT/RR^{0.33}) and the Mitchell et al. formula as well on traces that didn't corrected manually.

The rats were randomly divided into two groups, the first group (n=3) was slowly (˜1 min) administered intravenously 0.3 ml of 50 mg/kg MWL001. The second group (n=3) was intravenously administered QND 30 mg/kg/hr via syringe injection pump at 2.29 ml/hr rate. Additional rats were administered with 166 mg/kg/hr MWL intravenously via syringe injection pump at 2.29 ml/hr rate. Additional rats were administered saline as control.

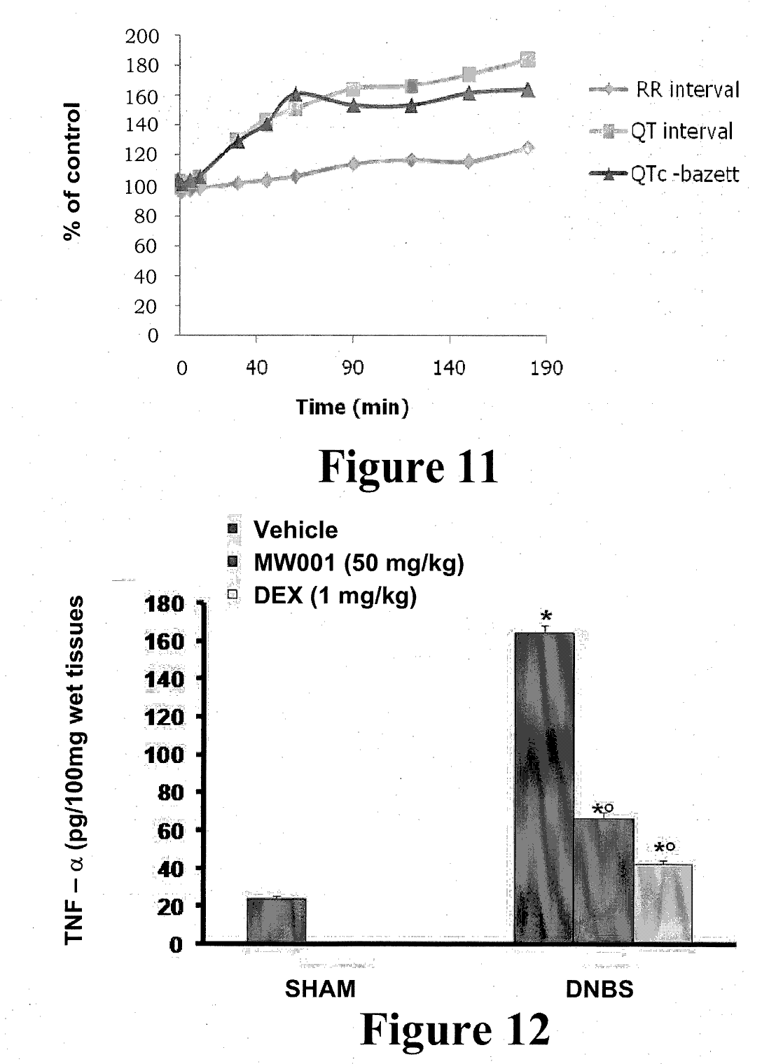

In all the animals the initial blood pressure (BP) was in the range of 120/80 and was maintained in this range. Representative data analysis presented in the figures below shows the data analysis method and ECG component identification. As shown, the injection of high concentrations of MWL (50-166 mg/kg) did not result in prolongation of the QTc interval in the two hr period after injection (see FIG. 10). Further, by comparing FIGS. 10 and 11, it is shown that no cardiotoxic effect of MWL is found in the rats' cardiovascular system in high doses and short term of 2-3 hr compared to Quinidine.

### Efficacy Studies

### a. Colitis Model-Oral Administration

In this study, the effect of MWL001 on pro-inflammatory mediators in DNBS-induced experimental colitis in mice was evaluated. In particular it was observed that there was no expression of TNF-α, CD4, TGF-13, CD25 in the colon tissue from sham-treated mice. MWL001 and DEX were administered orally and daily starting at six hours after the DNBS challenge.

Colitis was induced with a very low dose of 2,4-dinitrobenzene sulfonic acid (DNBS) (4 mg per mouse) by using a modification of the method first described in mice. In preliminary experiments, this dose of DNBS was found to induce reproducible colitis without mortality. Mice were anesthetized by Enflurane. DNBS (4 mg in 100 μl of 50% ethanol) was injected into the rectum through a catheter inserted 4.5 cm proximally to the anus. The carrier alone (100 μl of 50% ethanol) was administered in control experiments. Thereafter, the animals were kept for 15 minutes in a Trendelenburg position to avoid reflux. After colitis and sham-colitis induction, the animals were observed for three days. On Day four, the animals were weighed and anaesthetized with chloral hydrate, and their abdomen was opened by a midline incision. The colon was removed, freed from surrounding tissues, opened along the antimesenteric border and processed for histology.

TNF-α and IL-6 levels were evaluated and colon tissues collected at four days after DNBS administration. Briefly, portions of terminal colon were homogenized as previously described in phosphate-buffered saline (PBS, ICN Biomedicals, Milan, Italy) containing two mmol/L of phenyl-methyl sulfonyl fluoride (PMSF, Sigma Chemical Co.). The assay was carried out using a colorimetric, commercial kit (R&D system Milan, Italy) according to the manufacturer instructions. All cytokines determinations were performed in duplicate serial dilutions.

At four days after DNBS administration, colon tissues were fixed in 10% (w/v) PBS-buffered formaldehyde and 7 μm sections were prepared from paraffin embedded tissues. After deparaffinization, endogenous peroxidase was quenched with 0.3% (v/v) hydrogen peroxide in 60% (v/v) methanol for 30 min. The sections were permeabilized with 0.1% (w/v) Triton X-100 in PBS for 20 min. Non-specific adsorption was minimized by incubating the section in 2% (v/v) normal goat serum in PBS for 20 min. Endogenous biotin or avidin binding sites were blocked by sequential incubation for 15 min with biotin and avidin (Vector Laboratories, Burlingame, Calif.), respectively. Sections were incubated overnight with: 1) purified polyclonal antibody directed towards CD4 (Santa Cruz Biotechnology, 1:500 in PBS, v/v) or 2) with purified anti-CD25 (Santa Cruz Biotecnology 1:500 in PBS, w/v) or 3) with anti- TNF-α polyclonal antibody (Santa Cruz Biotechnology, N-19:sc-1350, 1:500 in PBS, v/v) or 4) with anti-TGF-β polyclonal antibody (Santa Cruz, 1:500 in PBS, v/v).

Sections were washed with PBS, and incubated with a secondary antibody. Specific labelling was detected with a biotin-conjugated goat anti-rabbit IgG and avidin-biotin peroxidase complex (Vector Laboratories, Burlingame, Calif.).

All of the procedures related to animal handling, care, and the treatment in this study were performed according to the guidelines approved by the Institutional Animal Care and Use Committee (IACUC) following the guidance of the Association for Assessment and Accreditation of Laboratory Animal Care (AAALAC). At the time of routine monitoring, the animals were checked for any effects of Colitis and treatments on normal behavior such as mobility, food and water consumption (by looking only), body weight gain/loss (body weights were measured daily), eye/hair matting and any other abnormal effect. Death and observed clinical signs were recorded on the basis of the numbers of animals within each subset.

### Results

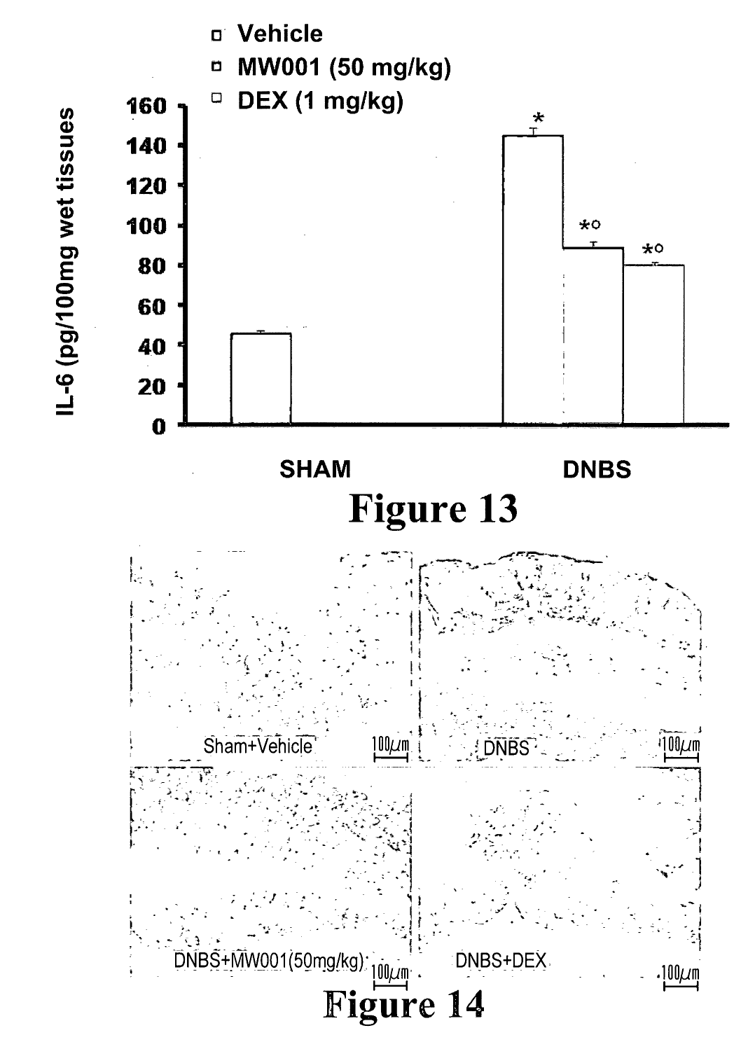

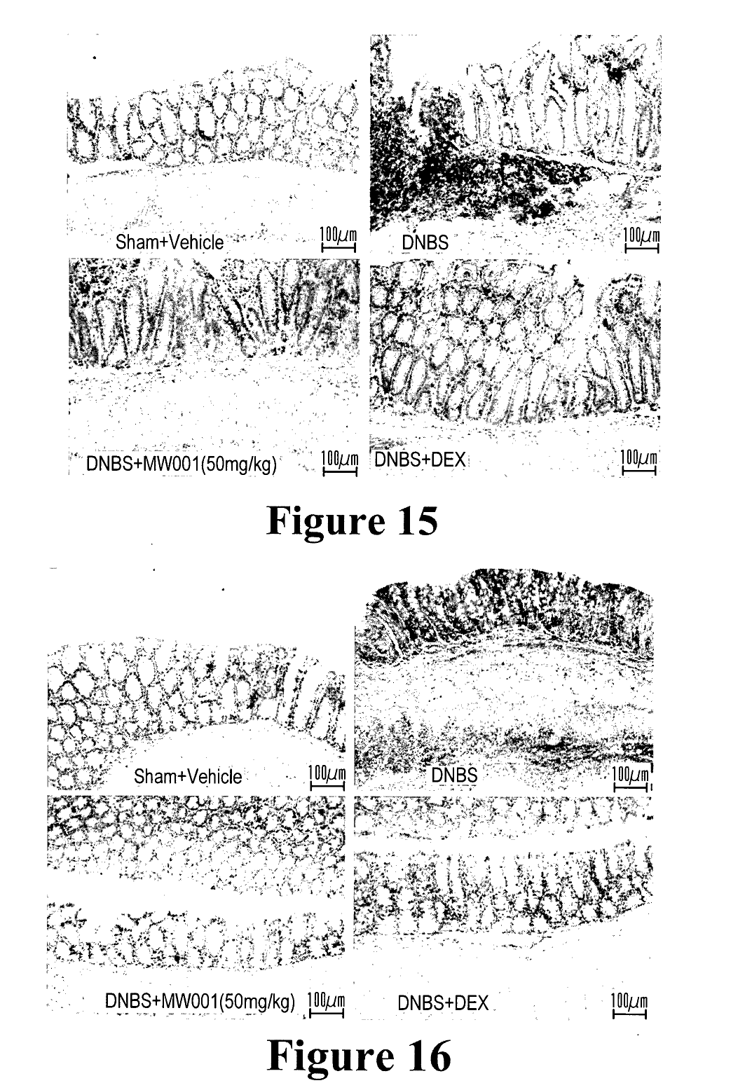

Four days after intra-colonic administration of DNBS, positive staining for TNF-α, TGF-β, CD25 and CD4 were observed in the inflammatory cells in the submucosa of colon section from DNBS-treated mice. The results of the TNF-α expression and levels of the colon from all of the experimental groups at day four are shown in FIG. 12. The IL-6 levels of the colon from all of the experimental groups at day four are shown in FIG. 13. The representative immunolocalization of TGF-β expression in the colon tissues, four days after the administration of DNBS, is shown in FIG. 14. The representative immunolocalization of CD25 expression in the colon tissues, four days after the administration of DNBS, is shown in FIG. 15. The representative immunolocalization of CD4 expression in the colon tissues, four days after the administration of DNBS, is shown in FIG. 16.

### Conclusions

The oral treatment with MWL001 (50 mg/kg), similarly to DEX treatment, significantly reduced the expression of TNF-α, TGF-β, CD25 and CD4. Four days after colitis induced was by DNBS, a significant increase of TNF-α and IL-6 levels was observed in the colon tissues. MWL001 oral treatment (50 mg/kg), similarly to DEX treatment, resulted in a significant reduction of the TNF-α and IL-6 colon levels.

### Dermatitis Model

An allergic form of contact dermatitis can be induced in mice by repeated epicutaneous application of the chemical sensitizer oxazolone. This leads to local augmented thickness and weight increase of the skin area where the rechallenge was applied; from the histological point of view, oxazolone-induced dermatitis is characterized by severe mononuclear cell infiltration of the dermis with in situ production of the type 1 proinflammatory cytokines TNF-alpha, IL-1alpha and IFN-gamma. These well-defined mechanistic pathways of inflammatory skin damage make oxazolone-dermatitis a useful in vivo tool for pathogenic studies and a pharmacodynamic parameter to screen drugs of potential utility in counteracting type 1 cytokine dependent cutaneous immunoinflammatory responses such as those found in some forms of bullous disorders, cutaneous vasculitis and psoriasis.

The efficacy of MWL001 in the treatment of Murine oxazolone-induced allergic dermatitis was evaluated. MWL001 and DEX were administered one hour after the second challenge with oxazolone.

The mice were sensitized on day 0 by a single application of 10 ∞l of 2% oxazolone (Sigma Chimica, Milan, Italy) in ethanol to the inner and outer surface of the left ear. The disease was elicited by local rechallenge on day 7 with 15% oxazolone. The right ear was treated with the vehicle of oxazolone (acetone). Eighteen hours after sensitization the mice are sacrificed under ether anesthesia and both the right and left ears were excised.

Inflammation was assessed as the percentage increase in ear thickness and/or ear weight in the treated left ear vs. the vehicle-treated right ear. Ear thickness was measured with a digital calliper. The extent of the inflammation was quantified as follows: ear swelling (%)=100×(a−b)/b, where a is thickness/weight of the left (treated) ear and b is the thickness/weight of the right (untreated) ear.

Ear tissue samples were fixed with 4% formaldehyde in phosphate-buffered saline (PBS) and embedded in paraffin. Thereafter, 6-μm sections were deparaffinized with xylene, stained with hematoxylin-eosin and observed with a Zeiss microscope (Jena, Germany).

Digital images were captured by AxioVision software (Carl Zeiss Vision, Munich, Germany) and assembled using Illustrator 9.0 (Adobe Systems, Mountain View, Calif.) on a Windows platform.

All the procedures related to animal handling, care, and the treatment in this study were performed according to the guidelines approved by the Institutional Animal Care and Use Committee (IACUC) following the guidance of the Association for Assessment and Accreditation of Laboratory Animal Care (AAALAC). At the time of routine monitoring, the animals were checked for any effects of Colitis and treatments on normal behavior such as mobility, food and water consumption (by looking only), body weight gain/loss (body weights were measured daily), eye/hair matting and any other abnormal effect. Death and observed clinical signs were recorded on the basis of the numbers of animals within each subset.

### Results and Conclusions

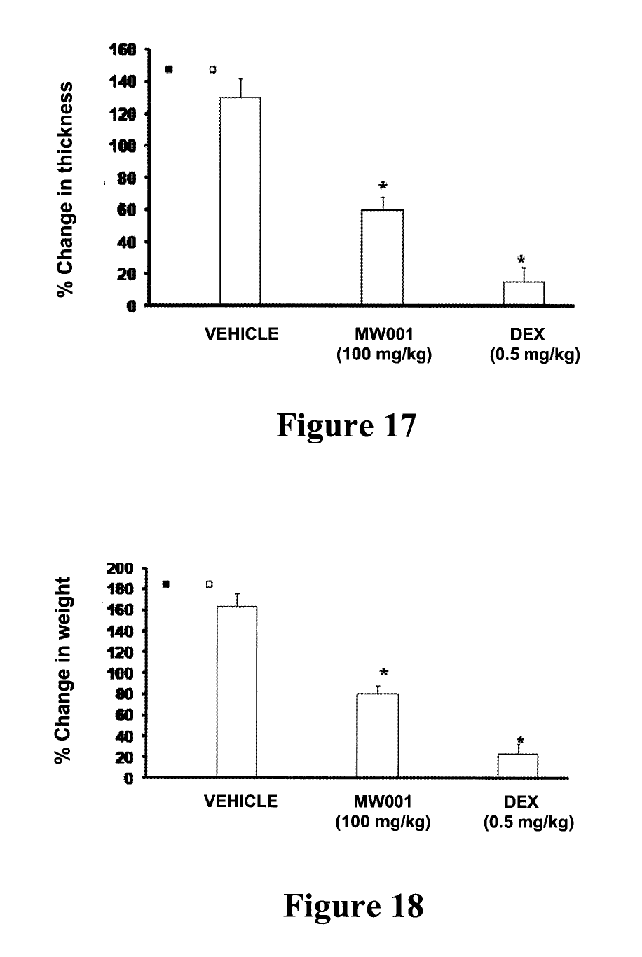

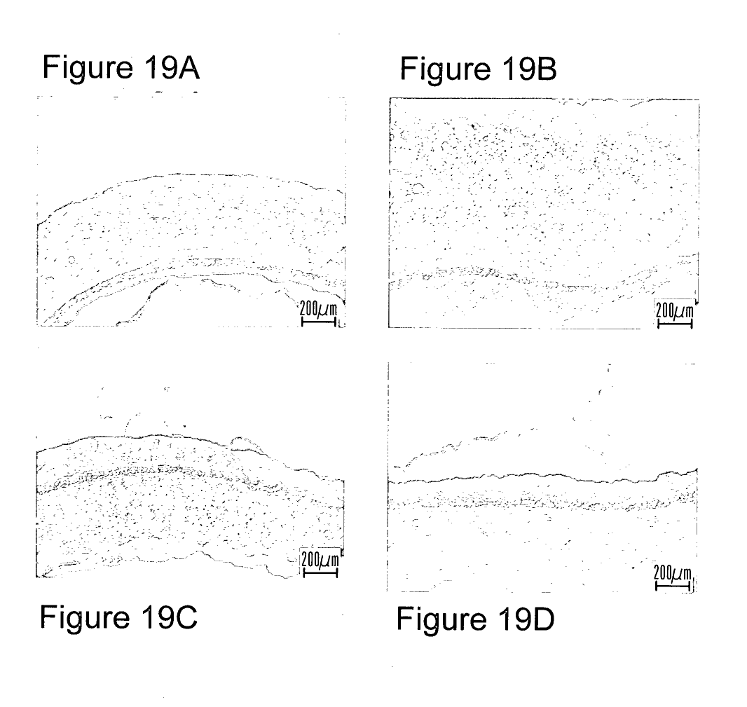

The results of the ear thickness at eighteen hours after sensitization from all the experimental groups are shown in FIG. 17. The results of the ear weight at eighteen hours after sensitization from all the experimental groups are shown in FIG. 18. The representative hematoxylin/eosin-stained sections of the ear tissues at eighteen hours after sensitization are shown in FIG. 19.

Oxazalone treatment of sensitized animals produced a large increase in both ear thickness and ear weight, showing that the oxazalone induces inflammation. The treatment with MWL001 reduced the oxazolone-induced inflammation, as shown by both decreased ear thickness and decreased ear weight. Hematoxylin and eosin stained sections demonstrated a marked increase in ear thickness with an abundance of inflammatory cells in both epidermis and dermis in oxazolone-treated animals, changes that were markedly reduced by MWL001 treatment, similarly to DEX treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-40. (canceled)

41. A method for treating dermatitis in a subject in need comprising the step of administering the subject in need docosa-4,7,10,13,16,19-hexaenoic acid linked to a hydroxyproline, thereby treating dermatitis in the subject.

42. A method of synthesizing 1-docosa-4,7,10,13,16,19-hexaenoyl-4-hydroxy-pyrrolidine-2-carboxylic acid comprising:
mixing tetrahydrofurane with decosahexanoic acid;
adding triethylcloroformate;
adding triethylamine;
stirring and filtering;
adding a solution of hydroxyproline and NaOH in water;
adding strong acid;
adding hexane;
collecting organic layer; and drying

43. The method according to claim 42, wherein the step of stirring following the addition of the hydroxyproline and NaOH in water solution, is for at 12 hours.

44. The method according to claim 42, wherein the step of drying is over anhydrous sulfate.

45. A chemical conjugate comprising a first moiety and a second moiety covalently linked therebetween, wherein said second moiety is derived from docosa-4,7,10,13,16,19-hexaenoic acid, and wherein said first moiety is derived from a therapeutically active agent or a derivative thereof, each independently having a functional group for forming a covalent bond with said second moiety, with the proviso that said first moiety is not hydroxyproline, the chemical conjugate being a cyclooxygenase-2 (COX-2) inhibitor.

46. The chemical conjugate of claim 45, being further a 5-lipoxygenase (5-LOX) inhibitor.

47. The chemical conjugate of claim 45, wherein said functional group is selected from the group consisting of hydroxy, amine, carboxy and amide.

48. The chemical conjugate of claim 45, wherein said first moiety and said second moiety are covalently bound via a bond selected from the group consisting of an amide bond and an ester bond.

49. The chemical conjugate of claim 45, wherein said therapeutically active agent is an anti-inflammatory agent or a cyclooxygenase (COX) inhibitor or a non-steroidal anti-inflammatory drug (NSAID).

50. The chemical conjugate of claim 45, wherein said therapeutically active agent is selected from the group consisting of: 5-hydroxy-indol-3-yl-acetic acid, 2-amino-nicotinic acid, salicyclic acid, mesalazine and quercetin.

51. A chemical conjugate comprising a first moiety and a second moiety covalently linked therebetween, wherein said second moiety is derived from y-linolenic acid, and wherein said first moiety is derived from a therapeutically active agent or a derivative thereof, each independently having a functional group for forming a covalent bond with said second moiety, with the proviso that said first moiety is not hydroxyproline or taurine, the chemical conjugate being a cyclooxygenase-2 (COX-2) inhibitor.

52. The chemical conjugate of claim 51, being further a 5-lipoxygenase (5-LOX) inhibitor.

53. The chemical conjugate of claim 51, wherein said functional group is selected from the group consisting of hydroxy, amine, carboxy and amide.

54. The chemical conjugate claim 51, wherein said first moiety and said second moiety are covalently bound via a bond selected from the group consisting of an amide bond and an ester bond.

55. The chemical conjugate of claim 51, wherein said therapeutically active agent is an anti-inflammatory agent, a cyclooxygenase (COX) inhibitor, a non-steroidal anti-inflammatory drug (NSAID) or selected from the group consisting of salicyclic acid and mesalazine.

56. A chemical conjugate comprising a first moiety and a second moiety covalently linked therebetween, wherein said first moiety is derived from a compound selected from the group consisting of 5-hydroxy-indol-3-yl-acetic acid, 2-amino-nicotinic acid, salicyclic acid, mesalazine, quercetin and resveratrol, and wherein said second moiety is derived from a fatty acid.

57. The chemical conjugate of claim 56, wherein said first moiety and said second moiety are covalently linked therebetween via a bon selected from the group consisting of an ester bond and an amide bond and the fatty acid is docosa-4,7,10,13,16,19-hexaenoic acid or γ-linolenic acid.

58. A chemical conjugate selected from the group consisting of:
N-(docosa-4,7,10,13,16,19-hexaenoyl)-5-hydroxy-indol-3-yl-aceticacid (MWL004);
N-(docosa-4,7,10,13,16,19-hexaenoyl)-2-amino-nicotinic acid (MWL005);
N-(docosa-4,7,10,13,16,19-hexaenoyl)-2-amino-phenyl-acetic acid (MWL006);
N-oleoyl-5-hydroxy-indol-3-yl-acetic acid (MWL007);
N-oleoyl-2-amino-nicotinic acid (MWL008);
O-oleoyl-salicylic acid (MWL009);
O-(docosa-4,7,10,13,16,19-hexaenoyl)-salicylic acid (MWL013);
O-(y-linolenoyl)-salicylic acid (MWL014);
N-(y-linolenoyl)-5-amino-salicylic acid (MWL015);
N-(docosa-4,7,10,13,16,19-hexaenoyl)-5-amino-salicylic acid (MWL016);
N-oleoyl-5-amino-salicylic acid (MWL017); and
N-docosa-4,7,10,13,16,19-hexaenoyl-taurine (MWL002).

59. A pharmaceutical composition comprising the chemical conjugate of claim 45 and a pharmaceutically acceptable carrier.

60. A method of treating an inflammatory disease or disorder, the method comprising administering to a subject in need thereof an effective amount of the chemical conjugate of claim 45.

61. The method of claim 60, wherein said inflammatory disease or disorder is selected from the group consisting of Alzheimer's disease, Parkinson's disease, asthma, osteoarthritis, rheumatoid arthritis, pain associated with inflammation, primary dysmenorrhea, Crohn's disease and ulcerative colitis.
